SEQUENCE LISTING (20 sequences)

SEQ ID NO: 1 - protein, 219 aa, Homo sapiens
MSSHLVEPPPPLHNNNNNCEENEQSLPPPAGLNSSWVELPMNSSNGNDNGNGKNGGLEHVPSSSSIHNGDMEKILLDAQHESGQSSSRGSSHCDSPSPQEDGQIMFDVEMHTSRDHSSQSEEEVVEGEKEVEALKKSADWVSDWSSRPENIPPKEFHFRHPKRSVSLSMRKSGAMKKGGIFSAEFLKVFIPSLFLSHVLALGLGIYIGKRLSTPSASTY

SEQ ID NO: 2 - DNA, 3505 bp, Homo sapiens
  source 1..3505: mol_type = other DNA
cgtcaggggcaggggagggacggcgcaggcgcagaaaagggggcggcggactcggcttgttgtgttgctgcctgagtgccggagacggtcctgctgctgccgcagtcctgccagctgtccgacaatgtcgtcccacctagtcgagccgccgccgcccctgcacaacaacaacaacaactgcgaggaaaatgagcagtctctgcccccgccggccggcctcaacagttcctgggtggagctacccatgaacagcagcaatggcaatgataatggcaatgggaaaaatggggggctggaacacgtaccatcctcatcctccatccacaatggagacatggagaagattcttttggatgcacaacatgaatcaggacagagtagttccagaggcagttctcactgtgacagcccttcgccacaagaagatgggcagatcatgtttgatgtggaaatgcacaccagcagggaccatagctctcagtcagaagaagaagttgtagaaggagagaaggaagtcgaggctttgaagaaaagtgcggactgggtatcagactggtccagtagacccgaaaacattccacccaaggagttccacttcagacaccctaaacgttctgtgtctttaagcatgaggaaaagtggagccatgaagaaagggggtattttctccgcagaatttctgaaggtgttcattccatctctcttcctttctcatgttttggctttggggctaggcatctatattggaaagcgactgagcacaccctctgccagcacctactgagggaaaggaaaagcccctggaaatgcgtgtgacctgtgaagtggtgtattgtcacagtagcttatttgaacttgagaccattgtaagcatgacccaacctaccaccctgtttttacatatccaattccagtaactctcaaattcaatattttattcaaactctgttgaggcattttactaaccttataccctttttggcctgaagacattttagaatttcctaacagagtttactgttgtttagaaatttgcaagggcttcttttccgcaaatgccaccagcagattataattttgtcagcaatgctattatctctaattagtgccaccagactagacctgtatcattcatggtataaattttactcttgcaacataactaccatctctctcttaaaacgagatcaggttagcaaatgatgtaaaagaagctttattgtctagttgttttttttcccccaagacaaaggcaagtttccctaagtttgagttgatagttattaaaaagaaaacaaaacaaaaaaaaaaggcaaggcacaacaaaaaaatatcctgggcaataaaaaaaatattttaaaccagctttggagccacttttttgtctaagcctcctaatagcgtcttttaatttataggaggcaaactgtataaatgataggtatgaaatagaataagaagtaaaatacatcagcagattttcatactagtatgttgtaatgctgtcttttctatggtgtagaatctttctttctgataaggaacgtctcaggcttagaaatatatgaaattgctttttgagatttttgcgtgtgtgtttgatattttttacgataattagctgcatgtgaatttttcatgaccttctttacattttttattttttatttctttatttttttttctctaagaagaggctttggaatgagttccaatttgtgatgttaatacaggcttcttgttttaggaagcatcacctatactctgaagcctttaaactctgaagagaattgtttcagagttattccaagcacttgtgcaacttggaaaaacagacttgggttgtgggaacagttgacagcgttctgaaaagatgccatttgtttccttctgatctctcactgaataatgtttactgtacagtcttcccaaggtgattcctgcgactgcaggcactggtcattttctcatgtagctgtcttttcagttatggtaaactcttaaagttcagaacactcaacagattccttcagtgatatacttgttcgttcatttctaaaatgtgaagctttaggaccaaattgttagaaagcatcaggatgaccagttatctcgagtagattttcttggatttcagaacatctagcatgactctgaaggataccacatgttttatatataaataattactgtttatgatatagacattgatattgactatttagagaaccgttgttaattttaaaactagcaatctataaagtgcaccaggtcaacttgaataaaaacactatgacagacaggtttgccagtttgcagaaactaactcttttctcacatcaacatttgtaaaattgatgtgttatagtggaaaataacatatagattaaacaaaatttttatcttttttcaagaatatagctggctatctttaagaaagatgatatatcctagttttgaaagtaattttcttttttctttctagcatttgatgtctaaataattttggacatctttttcctagaccatgtttctgtcttactcttaaacctggtaacacttgatttgccttctataacctatttatttcaagtgttcatatttgaatttctttgggaagaaagtaaatctgatggctcactgatttttgaaaagcctgaataaaattggaaagactggaaagttaggagaactgactagctaaactgctacagtatgcaatttctattacaattggtattacaggggggaaaagtaaaattacactttacctgaaagtgacttcttacagctagtgcattgtgctctttccaagttcagcagcagttctatcagtggtgccactgaaactgggtatatttatgatttctttcagcgttaaaaagaaacatagtgttgccctttttcttaaagcatcagtgaaattatggaaaattacttaaaacgtgaatacatcatcacagtagaatttattatgagagcatgtagtatgtatctgtagccctaacacatgggatgaacgttttactgctacacccagatttgtgttgaacgaaaacattgtggtttggaaaggagaattcaacaattaatagttgaaattgtgaggttaatgtttaaaaagctttacacctgtttacaatttggggacaaaaaggcaggcttcatttttcatatgtttgatgaaaactggctcaagatgtttgtaaatagaatcaagagcaaaactgcacaaacttgcacattggaaagtgcaacaagttcccgtgattgcagtaaaaatatttactattctaaaaaaatgagaattgaagacttagccagtcagataagttttttcatgaacccgttgtggaaattattggaattaactgagccaaagtgattatgcattcttcatctattttagttagcactttgtatcgttatatacagtttacaatacatgtataacttgtagctataaacattttgtgccattaaagctctcacaaaactttaaaaa

SEQ ID NO: 3 - protein, 117 aa, Homo sapiens
MKFQYKEDHPFEYRKKEGEKIRKKYPDRVPVIVEKAPKARVPDLDKRKYLVPSDLTVGQFYFLIRKRIHLRPEDALFFFVNNTIPPTSATMGQLYEDNHEEDYFLYVAYSDESVYGK

SEQ ID NO: 4 - DNA, 1885 bp, Homo sapiens
  source 1..1885: mol_type = other DNA
cagctctagcgaaaagccgccggtatttctccatctggctctcctctacctccaggcaggctcacccgagatccccgccccgaaccccccctgcacactcggcccagcgctgttgcccccggagcggacgtttctgcagctattctgagcacaccttgacgtcggctgagggagcgggacagggtcagcggcgaaggaggcaggccccgcgcggggatctcggaagccctgcggtgcatcatgaagttccagtacaaggaggaccatccctttgagtatcggaaaaaggaaggagaaaagatccggaagaaatatccggacagggtccccgtgattgtagagaaggctccaaaagccagggtgcctgatctggacaagaggaagtacctagtgccctctgaccttactgttggccagttctacttcttaatccggaagagaatccacctgagacctgaggacgccttattcttctttgtcaacaacaccatccctcccaccagtgctaccatgggccaactgtatgaggacaatcatgaggaagactattttctgtatgtggcctacagtgatgagagtgtctatgggaaatgagtggttggaagcccagcagatgggagcacctggacttgggggtaggggaggggtgtgtgtgcgcgacatggggaaagagggtggctcccaccgcaaggagacagaaggtgaagacatctagaaacattacaccacacacaccgtcatcacattttcacatgctcaattgatattttttgctgcttcctcggcccagggagaaagcatgtcaggacagagctgttggattggctttgatagaggaatggggatgatgtaagtttacagtattcctggggtttaattgttgtgcagtttcatagatgggtcaggaggtggacaagttggggccagagatgatggcagtccagcagcaactccctgtgctcccttctctttgggcagagattctatttttgacatttgcacaagacaggtagggaaaggggacttgtggtagtggaccatacctggggaccaaaagagacccactgtaattgatgcattgtggcccctgatcttccctgtctcacacttcttttctcccatcccggttgcaatctcactcagacatcacagtaccaccccaggggtggcagtagacaacaacccagaaatttagacagggatctcttacctttggaaaataggggttaggcatgaaggtggttgtgattaagaagatggttttgttattaaatagcattaaactggaattgacaagagtgttgagcatccctgtctaacctgctctttctctttggtgccccttatctcaccccttccttggaatttaataagtctcaggcatttccaattgtagactaaaaccactcttagcatctcctctagtattttccatgtatcaggacagaggtgtcttatgtagggagggggcaagtatgaagtaaggtaattatatactactctcattcaggattcttgctcccatgctgctgtcccttcaggctcacatgcacaggaatgctacatgatggccagctgcttccctccttggttatcatccactgcagctgctagttagaaaggtttggagggatgacttttagtaaatcatggggattttattgatttattttcacttttgggattttgtggggtgggagtggggagcaggaattgcactcagacatgacatttcaattcatctctgctaatgaaaagggttctttctcttgggggaaatgtgtgtgtcagttctgtcagctgcaagttcttgtataatgaagtcaatgccatcaggccaaggaaataaaataattgcttaccttaaaaaaaaaaaaaaaaaaaa

SEQ ID NO: 5 - DNA, 20 bp, synthetic construct
  misc_feature 1..20: note = Synthesised polynucleotide
  source 1..20: mol_type = other DNA
aggacaagagaaataaggcc

SEQ ID NO: 6 - DNA, 30 bp, synthetic construct
  misc_feature 1..30: note = Synthesised polynucleotide
  source 1..30: mol_type = other DNA
taagaagaggaattgaacctctgactgtaa

SEQ ID NO: 7 - DNA, 23 bp, synthetic construct
  misc_feature 1..23: note = Synthesised polynucleotide
  source 1..23: mol_type = other DNA
tttttgtgtgctctcccaggtct

SEQ ID NO: 8 - DNA, 19 bp, synthetic construct
  misc_feature 1..19: note = Synthesised polynucleotide
  source 1..19: mol_type = other DNA
tggtcactggttggttggc

SEQ ID NO: 9 - DNA, 28 bp, synthetic construct
  misc_feature 1..28: note = Synthesised polynucleotide
  source 1..28: mol_type = other DNA
ttcacaaagcgccttcccccgtaaatga

SEQ ID NO: 10 - DNA, 29 bp, synthetic construct
  misc_feature 1..29: note = Synthesised polynucleotide
  source 1..29: mol_type = other DNA
ccctgaactgcagatcaccaatgtggtag

SEQ ID NO: 11 - DNA, 23 bp, synthetic construct
  misc_feature 1..23: note = Synthesised polynucleotide
  source 1..23: mol_type = other DNA
ttggatgcacaacatgaatcagg

SEQ ID NO: 12 - DNA, 23 bp, synthetic construct
  misc_feature 1..23: note = Synthesised polynucleotide
  source 1..23: mol_type = other DNA
tcttctgactgagagctatggtc

SEQ ID NO: 13 - DNA, 21 bp, synthetic construct
  misc_feature 1..21: note = Synthesised polynucleotide
  source 1..21: mol_type = other DNA
gacgccttattcttctttgtc

SEQ ID NO: 14 - DNA, 22 bp, synthetic construct
  misc_feature 1..22: note = Synthesised polynucleotide
  source 1..22: mol_type = other DNA
catgattgtcctcatacagttc

SEQ ID NO: 15 - DNA, 20 bp, synthetic construct
  misc_feature 1..20: note = Synthesised polynucleotide
  source 1..20: mol_type = other DNA
gtttgtggataagacagtcc

SEQ ID NO: 16 - DNA, 20 bp, synthetic construct
  misc_feature 1..20: note = Synthesised polynucleotide
  source 1..20: mol_type = other DNA
gaagccaaaagtgttctctc

SEQ ID NO: 17 - DNA, 19 bp, synthetic construct
  misc_feature 1..19: note = Synthesised polynucleotide
  source 1..19: mol_type = other DNA
ttccccttggccatcaaga

SEQ ID NO: 18 - DNA, 20 bp, synthetic construct
  misc_feature 1..20: note = Synthesised polynucleotide
  source 1..20: mol_type = other DNA
accagctcctggctcattgt

SEQ ID NO: 19 - DNA, 20 bp, synthetic construct
  misc_feature 1..20: note = Synthesised polynucleotide
  source 1..20: mol_type = other DNA
gtcctctcccaagtccacac

SEQ ID NO: 20 - DNA, 19 bp, synthetic construct
  misc_feature 1..19: note = Synthesised polynucleotide
  source 1..19: mol_type = other DNA
gggagaccaaaagcttcat

(19) United States
(12) Patent Application Publication
(10) Pub. No.: US 2024/0066098 A1
(43) Pub. Date: Feb. 29, 2024

(54) COMPOSITIONS AND METHODS FOR THE TREATMENT OR PREVENTION OF NEURODEGENERATIVE DISORDERS

(71) Applicant: Northern Sydney Local Health District, St. Leonards, NSW (AU)

(72) Inventors: Carolyn M. Sue, St. Leonards (AU); Brianada Koentjoro, Macquarie Park (AU); Jin-Sung Park, Epping (AU)

(21) Appl. No.: 18/069,546

(22) Filed: Dec. 21, 2022

Related U.S. Application Data
(63) Continuation of application No. 16/854,717, filed on Apr. 21, 2020, now abandoned.
Application No. 16/854,717 is a continuation of application No. 16/215,129, filed on Dec. 10, 2018, now abandoned.
Application No. 16/215,129 is a continuation of application No. 15/301,233, filed on Sep. 30, 2016, now abandoned.

(30) Foreign Application Priority Data
Apr. 16, 2014 (AU) 2014901398

(51) Int. Cl. A61K 38/17; A61K 48/00; G01N 33/50; G01N 33/68

(52) U.S. Cl. CPC A61K 38/1761; A61K 38/1709; A61K 48/005; G01N 33/5023; G01N 33/6896; A61K 48/00; G01N 2500/10; G01N 2800/28

(57) ABSTRACT

The present invention provides a method for the prevention or treatment of a neurodegenerative disorder in a subject, comprising administering to the subject a therapeutically effective amount of an agent that increases Nix-mediated mitophagy in a cell. Also provided is a method for identifying a compound useful for the prevention or treatment of a neurodegenerative disorder in a subject.

This application is a continuation of U.S. patent application Ser. No. 16/854,717, filed Apr. 21, 2020, which is a continuation of U.S. patent application Ser. No. 16/215,129, filed Dec. 10, 2018, which is a continuation of abandoned U.S. patent application Ser. No. 15/301,233, filed Sep. 30, 2016 which is a 371 of International Application No. PCT/AU2015/000194 filed Apr. 7, 2015, which claims the benefit of and priority to Australian Provisional Application No. 2014901398, filed 16 Apr. 2014, the entire disclosures of which are hereby incorporated by reference herein.

### FIELD

This invention relates to methods for treating or preventing neurodegenerative disorders by administering an agent that activates Nix-mediated mitophagy.

### SEQUENCE LISTING

The text of the computer readable sequence listing filed herewith, titled “SPRUS-35045-304_SQL”, created Dec. 20, 2022, having a file size of 30,600 bytes, is hereby incorporated by reference in its entirety.

### BACKGROUND

Neurodegenerative diseases are a large group of disabling disorders of the nervous system which are characterised by damage and death of neuronal subtypes. Mitochondrial dysfunction is regarded as a putative causative factor in a variety of neurodegenerative diseases including Parkinson's disease, Alzheimer's disease, Huntington's disease and mitochondrial disease.

Mitochondria are essential organelles that provide cellular energy through oxidative phosphorylation, regulate calcium homeostasis and cell death. However, mitochondria are also the major source of cellular reactive oxygen species (ROS). Normal levels of ROS can be tolerated because of cellular anti-oxidants, whereas in pathological situations of mitochondrial respiratory defect, increased production of ROS exceeds the capability of antioxidant protection, causing damage to a various cellular components including mitochondria. The accumulation of this damage is considered to render mitochondria dysfunctional. Accordingly, the removal of dysfunctional or damaged mitochondria through autophagy, a process called mitophagy, is critical for maintaining proper cellular functions.

Parkinson's disease (PD) is caused by specific and progressive neuronal loss of mid-brain dopamine neurons. Dopamine is a chemical messenger responsible for transmitting signals between the substantia nigra and the corpus striatum. Loss of dopamine causes the nerve cells of the striatum to fire in an uncontrolled manner resulting in the cardinal clinical features of bradykinesia, resting tremor, rigidity and postural instability; features that can be severe and profoundly crippling.

Among the several causative genes identified in familial forms of PD, mutations in parkin, a gene that encodes a E3 ubiquitin ligase, represents the most common genetic cause of autosomal recessive early-onset PD. PD patients with parkin mutations exhibit typical parkinsonism with early onset, slow progression, early dystonia and L-dopa responsiveness. Moreover, a wide range of disease expressivity and penetrance is associated with Parkin-related PD.

Parkin has also been implicated in the quality control of mitochondria. Parkin, together with PTEN-induced putative kinase 1 (PINK1), a mitochondrial kinase, mediates the selective autophagic removal of damaged mitochondria. Accordingly, PD-associated mutations in parkin are associated with impaired mitophagy.

There is no cure for PD. Current therapy relies heavily on replenishing dopamine by giving patients oral doses of a dopaminergic agent like the dopamine precursor levodopa or a dopamine agonist. Such therapy can provide relief but is associated with diminishing therapeutic efficacy requiring increased dosages with continuing treatment which is associated with an increased risk of serious side effects. There is a profound need for additional therapies for PD.

### SUMMARY OF INVENTION

The present inventors have determined that activation of mitophagy mediated by Nix can prevent and treat a neurodegenerative disease or disorder. According to one aspect, the present invention provides a method for the prevention or treatment of a neurodegenerative disorder in a subject, comprising administering to the subject a therapeutically effective amount of an agent that increases Nix-mediated mitophagy in a cell.

In one embodiment, the agent increases the expression of a Nix polypeptide or fragment thereof, and/or a GABARAP-L1 polypeptide or fragment thereof in a cell.

In one embodiment, the agent comprises a Nix polypeptide or fragment thereof and/or a GABARAP-L1 polypeptide or fragment thereof.

In another embodiment, the agent comprises an expression vector encoding a Nix polypeptide or fragment thereof and/or a GABARAP-L1 polypeptide or fragment thereof.

In one embodiment, the cell is a neuron.

In one embodiment the neurodegenerative disorder comprises deficient mitophagy in neurons of the subject.

In one embodiment the neurodegenerative disorder is selected from the group comprising Parkinson's disease, Alzheimer's disease, Lewy body dementia, Creutzfeldt-Jakob disease, Huntington's disease, mitochondrial disease, multiple sclerosis or amyotrophic lateral sclerosis.

In one embodiment the neurodegenerative disorder is Parkinson's disease. In another embodiment, the Parkinson's diseases is early onset Parkinson's disease (EOPD).

In one embodiment the subject possesses a mutation in parkin and/or PINK1.

According to another aspect, the present invention provides a method for identifying an agent useful for the prevention or treatment of a neurodegenerative disorder in a subject comprising: (a) contacting a cell with an agent; and (b) detecting an increase in the biological activity or expression of a polypeptide associated with Nix-mediated mitophagy, or (c) detecting an increase in the expression of a polynucleotide encoding a polypeptide associated with Nix-mediated mitophagy in the cell relative to a control cell not contacted with the agent, wherein an agent that increases said activity or said expression is identified as useful for the treatment of a neurodegenerative disorder.

In one embodiment, the cell used in a method for identifying a compound useful for the prevention or treatment of a neurodegenerative disorder in a subject displays impaired Parkin-related mitophagy.

In one embodiment, the cell used in a method for identifying a compound useful for the prevention or treatment of a neurodegenerative disorder in a subject comprises a mutation in parkin and/or PINK1.

In one embodiment, the cell used in a method for identifying a compound useful for the prevention or treatment of a neurodegenerative disorder in a subject is isolated from a subject that has a neurodegenerative disorder or is at risk of having a neurodegenerative disorder.

In one embodiment, the cell used in a method for identifying a compound useful for the prevention or treatment of a neurodegenerative disorder in a subject is a stem cell, an inducible pluripotent stem cell (iPS cell), a progenitor cell, or any cell derived therefrom, fibroblast, olfactory neurosphere or neuron.

According to another aspect, the present invention provides a kit for treating a neurodegenerative disorder comprising a pharmaceutical composition comprising a therapeutically effective amount of an agent that increases Nix-mediated mitophagy in a cell, instructions for identifying a subject in need of such treatment, and directions for administering the pharmaceutical composition to the subject.

In one embodiment, the pharmaceutical composition comprises an agent that increases the expression of a Nix polypeptide or fragment thereof, and/or a GABARAP-L1 polypeptide or fragment thereof in a cell.

In one embodiment, the pharmaceutical composition comprises a Nix polypeptide or fragment thereof and/or a GABARAP-L1 polypeptide or fragment thereof.

In one embodiment, the pharmaceutical composition comprises an expression vector encoding a Nix polypeptide or fragment thereof and/or a GABARAP-L1 polypeptide or fragment thereof.

According to another aspect, the present invention provides a use of an agent that increases Nix-mediated mitophagy in a cell in the preparation of a medicament for the prevention or treatment of a neurodegenerative disorder.

According to another aspect, the present invention provides an agent that increases Nix-mediated mitophagy in a cell for use in the prevention or treatment of a neurodegenerative disease.

The present invention thus relates to at least the following series of numbered embodiments below:

Embodiment 1: A method for the prevention or treatment of a neurodegenerative disorder in a subject, comprising administering to the subject a therapeutically effective amount of an agent that increases Nix-mediated mitophagy in a cell.

Embodiment 2: A method according to embodiment 1, wherein the agent increases the biological activity or expression of a Nix polypeptide or fragment or variant or analog thereof, and/or a GABARAP-L1 polypeptide or fragment or variant or analog thereof in a cell.

Embodiment 3: A method according to embodiment 1 or 2 wherein the agent comprises a Nix polypeptide or fragment or variant thereof, and/or a GABARAP-L1 polypeptide or fragment or variant thereof.

Embodiment 4: A method according to any one of the preceding embodiments, wherein the agent comprises an expression vector encoding a Nix polypeptide or fragment or variant thereof, and/or a GABARAP-L1 polypeptide or fragment or variant thereof.

Embodiment 5: A method according to any one of the preceding embodiments, wherein the agent comprises an expression vector encoding a Nix polypeptide or fragment or variant thereof.

Embodiment 6: A method according to any one of the preceding embodiments, wherein the cell is a neuron or a neuronal precursor.

Embodiment 7: A method according to any one of the preceding embodiments, wherein the neurodegenerative disorder is associated with mitochondrial dysfunction.

Embodiment 8: A method according to any one of the preceding embodiments wherein the neurodegenerative disorder comprises impaired mitophagy.

Embodiment 9: A method according to any one of the preceding embodiments, wherein the neurodegenerative disorder is selected from the group comprising Parkinson's disease, Alzheimer's disease, Lewy body dementia, Creutzfeldt-Jakob disease, Huntington's disease, multiple sclerosis or amyotrophic lateral sclerosis.

Embodiment 10: A method according to any one of the preceding embodiments, wherein the neurodegenerative disorder is Parkinson's disease.

Embodiment 11: A method according to any one of the preceding embodiments, wherein said subject possesses a mutation in parkin and/or PINK1.

Embodiment 12: A method for identifying an agent useful for the prevention or treatment of a neurodegenerative disorder in a subject comprising: (a) contacting a cell with an agent; and (b) detecting an increase in the biological activity or expression of one or more polypeptides associated with Nix-mediated mitophagy in the cell relative to a control cell not contacted with the agent, or (c) detecting an increase in the expression of one or more polynucleotides encoding a polypeptide associated with Nix-mediated mitophagy in the cell relative to a control cell not contacted with the agent, wherein an agent that increases said activity or said expression is identified as useful for the treatment of a neurodegenerative disorder.

Embodiment 13: A method according to embodiment 12, wherein said one or more polynucleotides or said one or more polypeptides associated with Nix-mediated mitophagy includes Nix and/or GABARAP-L1.

Embodiment 14: A method according to embodiment 12 or 13, wherein the cell displays impaired Parkin-related mitophagy.

Embodiment 15: A method according to any one of embodiments 12-14, wherein the cell comprises a mutation in parkin and/or PINK1.

Embodiment 16: A method according to any one of embodiments 12-15, wherein the cell is isolated from a subject that has a neurodegenerative disorder or is at risk of having a neurodegenerative disorder.

Embodiment 17: A method according to any one of embodiments 12-16, wherein the cell is a fibroblast, olfactory neurosphere or neuron.

Embodiment 18: A kit for treating a neurodegenerative disorder comprising a pharmaceutical composition comprising a therapeutically effective amount of an agent that increases Nix-mediated mitophagy in a cell, instructions for identifying a subject in need of such treatment, and directions for administering the pharmaceutical composition to the subject.

Embodiment 19: A kit according to embodiment 18, wherein the pharmaceutical composition comprises an agent that increases the expression of a Nix polypeptide or fragment thereof, and/or a GABARAP-L1 polypeptide or fragment thereof in a cell.

Embodiment 20: A kit according to embodiment 18 or 19, wherein the pharmaceutical composition comprises a Nix polypeptide or fragment thereof and/or a GABARAP-L1 polypeptide or fragment thereof.

Embodiment 21: A kit according to any one of embodiments 18-20, wherein the pharmaceutical composition comprises an expression vector encoding a Nix polypeptide or fragment thereof and/or a GABARAP-L1 polypeptide or fragment thereof.

Embodiment 22: Use of an agent that increases Nix-mediated mitophagy in a cell in the preparation of a medicament for the prevention or treatment of a neurodegenerative disorder.

Embodiment 23: An agent that increases Nix-mediated mitophagy in a cell for use in the prevention or treatment of a neurodegenerative disease.

Embodiment 24: A use according to embodiment 22 or an agent according to embodiment 23, wherein the agent increases the biological activity or expression of a Nix polypeptide or fragment or variant or analog thereof, and/or a GABARAP-L1 polypeptide or fragment or variant or analog thereof in a cell.

Embodiment 25: A use according to embodiment 22 or an agent according to embodiment 23, wherein the agent comprises a Nix polypeptide or fragment or variant thereof, and/or a GABARAP-L1 polypeptide or fragment or variant thereof.

Embodiment 26: A use according to embodiment 22 or an agent according to embodiment 23, wherein the agent comprises an expression vector encoding a Nix polypeptide or fragment or variant thereof.

Embodiment 27: A use according to embodiment 22 or any one of embodiments 24-26, or an agent according to any one of embodiments 23-26, wherein the neurodegenerative disorder is associated with mitochondrial dysfunction.

Embodiment 28: A use according to embodiment 22 or any one of embodiments 24-27, or an agent according to any one of embodiments 23-27, wherein the neurodegenerative disorder comprises impaired mitophagy.

Embodiment 29: A use according to embodiment 22 or any one of embodiments 24-28, or an agent according to any one of embodiments 23-28, wherein the neurodegenerative disorder is selected from the group comprising Parkinson's disease, Alzheimer's disease, Lewy body dementia, Creutzfeldt-Jakob disease, Huntington's disease, multiple sclerosis or amyotrophic lateral sclerosis.

Embodiment 30: A use according to embodiment 29 or an agent according to embodiment 29, wherein the neurodegenerative disorder is Parkinson's disease.

Embodiment 31: A use according to embodiment 22 or any one of embodiments 24-30, or an agent according to any one of embodiments 23-30, wherein the neurodegenerative disorder is associated with a mutation in parkin and/or PINK1.

### BRIEF DESCRIPTION OF DRAWINGS

FIG. 1A-C shows mitochondrial function is preserved in cells isolated from an individual carrying a homozygous mutation in parkin but has no PD (1A). It also illustrates that cells isolated from an individual carrying a heterozygous mutation in parkin with PD are more vulnerable to a mitochondrial toxin such as rotenone (1B and C).

FIG. 2A-D shows mitophagy is normal in cells isolated from an individual (“Carrier”) carrying a homozygous mutation in parkin but has no PD.

FIG. 3A-D shows a lack of compensation on Parkin function in mitophagy and aberrant induction of autophagy in cells isolated from an individual carrying a homozygous mutation in parkin but has no PD.

FIG. 4A-B shows expression of Nix and GABARAP-L1 is elevated in cells isolated from an individual carrying a homozygous mutation in parkin but has no PD.

FIG. 5A-F shows Nix knockdown abrogated CCCP-induced mitophagy in cells isolated from an individual carrying a homozygous mutation in parkin but has no PD.

FIG. 6A-D shows the specific induction of expression of Nix in cells isolated from an individual carrying compound heterozygous mutations in parkin with PD.

FIG. 7A-B shows specific induction of Nix restores mitophagy in cells isolated from an individual carrying compound heterozygous mutations in parkin with PD. FIG. 7(B) also depicts restoration of mitophagy in cells isolated from an individual with PD carrying a homozygous mutation in PINK1.

FIG. 8A-B shows Nix knockdown in cells isolated from an individual carrying compound heterozygous mutations in parkin with PD and cells isolated from an individual carrying a homozygous mutation in PINK1 with PD abrogated restoration of CCCP-induced mitophagy by an agent which induces Nix Expression.

FIG. 9A-C shows augmented expression of Nix restores CCCP-induced mitophagy in cells isolated from an individual carrying compound heterozygous mutations in parkin with PD.

FIG. 10 shows over-expression of Nix rescues mitochondrial function in cells isolated from an individual carrying compound heterozygous mutations in parkin with PD and cells isolated from an individual carrying a homozygous mutation in PINK1 with PD.

### SEQUENCES REFERRED TO HEREIN

```
SEQ ID NO: 1: an amino acid sequence encoding a Nix polypeptide:
MSSHLVEPPP PLHNNNNNCE ENEQSLPPPA GLNSSWVELP MNSSNGNDNG NGKNGGLEHV
PSSSSIHNGD MEKILLDAQH ESGQSSSRGS SHCDSPSPQE DGQIMEDVEM HTSRDHSSQS
EEEVVEGEKE VEALKKSADW VSDWSSRPEN IPPKEFHFRH PKRSVSLSMR KSGAMKKGGI
FSAEFLKVFI PSLFLSHVLA LGLGIYIGKR LSTPSASTY
SEQ ID NO: 2: a nucleic acid sequence encoding a Nix polypeptide:
      1 cgtcaggggc aggggaggga cggcgcaggc gcagaaaagg gggcggcgga ctcggcttgt
    61 tgtgttgctg cctgagtgcc ggagacggtc ctgctgctgc cgcagtcctg ccagctgtcc
  121 gacaatgtcg tcccacctag tcgagccgcc gccgcccctg cacaacaaca acaacaactg
  181 cgaggaaaat gagcagtctc tgcccccgcc ggccggcctc aacagttcct gggtggagct
  241 acccatgaac agcagcaatg gcaatgataa tggcaatggg aaaaatgggg ggctggaaca
  301 cgtaccatcc tcatcctcca tccacaatgg agacatggag aagattcttt tggatgcaca
  361 acatgaatca ggacagagta gttccagagg cagttctcac tgtgacagcc cttcgccaca
  421 agaagatggg cagatcatgt ttgatgtgga aatgcacacc agcagggacc atagctctca
  481 gtcagaagaa gaagttgtag aaggagagaa ggaagtcgag gctttgaaga aaagtgcgga
  541 ctgggtatca gactggtcca gtagacccga aaacattcca cccaaggagt tccacttcag
  601 acaccctaaa cgttctgtgt ctttaagcat gaggaaaagt ggagccatga agaaaggggg
  661 tattttctcc gcagaatttc tgaaggtgtt cattccatct ctcttccttt ctcatgtttt
  721 ggctttgggg ctaggcatct atattggaaa gcgactgagc acaccctctg ccagcaccta
  781 ctgagggaaa ggaaaagccc ctggaaatgc gtgtgacctg tgaagtggtg tattgtcaca
  841 gtagcttatt tgaacttgag accattgtaa gcatgaccca acctaccacc ctgtttttac
  901 atatccaatt ccagtaactc tcaaattcaa tattttattc aaactctgtt gaggcatttt
  961 actaacctta tacccttttt ggcctgaaga cattttagaa tttcctaaca gagtttactg
1021 ttgtttagaa atttgcaagg gcttcttttc cgcaaatgcc accagcagat tataattttg
1081 tcagcaatgc tattatctct aattagtgcc accagactag acctgtatca ttcatggtat
1141 aaattttact cttgcaacat aactaccato tctctcttaa aacgagatca ggttagcaaa
1201 tgatgtaaaa gaagctttat tgtctagttg ttttttttcc cccaagacaa aggcaagttt
1261 ccctaagttt gagttgatag ttattaaaaa gaaaacaaaa caaaaaaaaa aggcaaggca
1321 caacaaaaaa atatcctggg caataaaaaa aatattttaa accagctttg gagccacttt
1381 tttgtctaag cctcctaata gcgtctttta atttatagga ggcaaactgt ataaatgata
1441 ggtatgaaat agaataagaa gtaaaataca tcagcagatt ttcatactag tatgttgtaa
1501 tgctgtcttt tctatggtgt agaatctttc tttctgataa ggaacgtctc aggcttagaa
1561 atatatgaaa ttgctttttg agatttttgc gtgtgtgttt gatatttttt acgataatta
1621 gctgcatgtg aatttttcat gaccttcttt acatttttta ttttttattt ctttattttt
1681 ttttctctaa gaagaggctt tggaatgagt tccaatttgt gatgttaata caggcttctt
1741 gttttaggaa gcatcaccta tactctgaag cctttaaact ctgaagagaa ttgtttcaga
1801 gttattccaa gcacttgtgc aacttggaaa aacagacttg ggttgtggga acagttgaca
1861 gcgttctgaa aagatgccat ttgtttcctt ctgatctctc actgaataat gtttactgta
1921 cagtcttccc aaggtgattc ctgcgactgc aggcactggt cattttctca tgtagctgtc
1981 ttttcagtta tggtaaactc ttaaagttca gaacactcaa cagattcctt cagtgatata
2041 cttgttcgtt catttctaaa atgtgaagct ttaggaccaa attgttagaa agcatcagga
2101 tgaccagtta tctcgagtag attttcttgg atttcagaac atctagcatg actctgaagg
2161 ataccacatg ttttatatat aaataattac tgtttatgat atagacattg atattgacta
2221 tttagagaac cgttgttaat tttaaaacta gcaatctata aagtgcacca ggtcaacttg
2281 aataaaaaca ctatgacaga caggtttgcc agtttgcaga aactaactct tttctcacat
2341 caacatttgt aaaattgatg tgttatagtg gaaaataaca tatagattaa acaaaatttt
2401 tatctttttt caagaatata gctggctatc tttaagaaag atgatatatc ctagttttga
2461 aagtaatttt cttttttctt tctagcattt gatgtctaaa taattttgga catctttttc
2521 ctagaccatg tttctgtctt actcttaaac ctggtaacac ttgatttgcc ttctataacc
2581 tatttatttc aagtgttcat atttgaattt ctttgggaag aaagtaaatc tgatggctca
2641 ctgatttttg aaaagcctga ataaaattgg aaagactgga aagttaggag aactgactag
2701 ctaaactgct acagtatgca atttctatta caattggtat tacagggggg aaaagtaaaa
2761 ttacacttta cctgaaagtg acttottaca gctagtgcat tgtgctcttt ccaagttcag
2821 cagcagttct atcagtggtg ccactgaaac tgggtatatt tatgatttct ttcagcgtta
2881 aaaagaaaca tagtgttgcc ctttttctta aagcatcagt gaaattatgg aaaattactt
2941 aaaacgtgaa tacatcatca cagtagaatt tattatgaga gcatgtagta tgtatctgta
3001 gccctaacac atgggatgaa cgttttactg ctacacccag atttgtgttg aacgaaaaca
3061 ttgtggtttg gaaaggagaa ttcaacaatt aatagttgaa attgtgaggt taatgtttaa
3121 aaagctttac acctgtttac aatttgggga caaaaaggca ggcttcattt ttcatatgtt
3181 tgatgaaaac tggctcaaga tgtttgtaaa tagaatcaag agcaaaactg cacaaacttg
3241 cacattggaa agtgcaacaa gttcccgtga ttgcagtaaa aatatttact attctaaaaa
3301 aatgagaatt gaagacttag ccagtcagat aagttttttc atgaacccgt tgtggaaatt
3361 attggaatta actgagccaa agtgattatg cattcttcat ctattttagt tagcactttg
3421 tatcgttata tacagtttac aatacatgta taacttgtag ctataaacat tttgtgccat
3481 taaagctctc acaaaacttt aaaaa
SEQ ID NO: 3: an amino acid sequence encoding a GABA(A) receptor-associated
protein like 1 (GABARAP-L1) polypeptide:
MKFQYKEDHP FEYRKKEGEK IRKKYPDRVP VIVEKAPKAR VPDLDKRKYL VPSDLTVGOF
YFLIRKRIHL RPEDALFFFV NNTIPPTSAT MGQLYEDNHE EDYFLYVAYS DESVYGK
SEQ ID NO: 4: a nucleic acid sequence encoding a GABA(A) receptor-associated
protein like 1 (GABARAP-L1) polypeptide:
      1 cagctctagc gaaaagccgc cggtatttct ccatctggct ctcctctacc tccaggcagg
    61 ctcacccgag atccccgccc cgaacccccc ctgcacactc ggcccagcgc tgttgccccc
  121 ggagcggacg tttctgcagc tattctgagc acaccttgac gtcggctgag ggagcgggac
  181 agggtcagcg gcgaaggagg caggccccgc gcggggatct cggaagccct gcggtgcatc
  241 atgaagttcc agtacaagga ggaccatccc tttgagtatc ggaaaaagga aggagaaaag
  301 atccggaaga aatatccgga cagggtcccc gtgattgtag agaaggctcc aaaagccagg
  361 gtgcctgatc tggacaagag gaagtaccta gtgccctctg accttactgt tggccagttc
  421 tacttcttaa tccggaagag aatccacctg agacctgagg acgccttatt cttctttgtc
  481 aacaacacca tccctcccac cagtgctacc atgggccaac tgtatgagga caatcatgag
  541 gaagactatt ttctgtatgt ggcctacagt gatgagagtg tctatgggaa atgagtggtt
  601 ggaagcccag cagatgggag cacctggact tgggggtagg ggaggggtgt gtgtgcgcga
  661 catggggaaa gagggtggct cccaccgcaa ggagacagaa ggtgaagaca totagaaaca
  721 ttacaccaca cacaccgtca tcacattttc acatgctcaa ttgatatttt ttgctgcttc
  781 ctcggcccag ggagaaagca tgtcaggaca gagctgttgg attggctttg atagaggaat
  841 ggggatgatg taagtttaca gtattcctgg ggtttaattg ttgtgcagtt tcatagatgg
  901 gtcaggaggt ggacaagttg gggccagaga tgatggcagt ccagcagcaa ctccctgtgc
  961 tcccttctct ttgggcagag attctatttt tgacatttgc acaagacagg tagggaaagg
1021 ggacttgtgg tagtggacca tacctgggga ccaaaagaga cccactgtaa ttgatgcatt
1081 gtggcccctg atcttccctg tctcacactt cttttctccc atcccggttg caatctcact
1141 cagacatcac agtaccaccc caggggtggc agtagacaac aacccagaaa tttagacagg
1201 gatctcttac ctttggaaaa taggggttag gcatgaaggt ggttgtgatt aagaagatgg
1261 ttttgttatt aaatagcatt aaactggaat tgacaagagt gttgagcatc cctgtctaac
1321 ctgctctttc tctttggtgc cccttatctc accccttcct tggaatttaa taagtctcag
1381 gcatttccaa ttgtagacta aaaccactct tagcatctcc tctagtattt tccatgtatc
1441 aggacagagg tgtcttatgt agggaggggg caagtatgaa gtaaggtaat tatatactac
1501 tctcattcag gattcttgct cccatgctgc tgtcccttca ggctcacatg cacaggaatg
1561 ctacatgatg gccagctgct tccctccttg gttatcatcc actgcagctg ctagttagaa
1621 aggtttggag ggatgacttt tagtaaatca tggggatttt attgatttat tttcactttt
1681 gggattttgt ggggtgggag tggggagcag gaattgcact cagacatgac atttcaattc
1741 atctctgcta atgaaaaggg ttctttctct tgggggaaat gtgtgtgtca gttctgtcag
1801 ctgcaagttc ttgtataatg aagtcaatgc catcaggcca aggaaataaa ataattgctt
1861 accttaaaaa aaaaaaaaaa aaaaa
SEQ ID NO: 5: aggacaagagaaataaggcc (mitochondrial DNA forward primer)
SEQ ID NO: 6: taagaagaggaattgaacctctgactgtaa (mitochondrial DNA reverse primer)
SEQ ID NO: 7: tttttgtgtgctctcccaggtct (nuclear DNA forward primer)
SEQ ID NO: 8: tggtcactggttggttggc (nuclear DNA reverse primer)
SEQ ID NO: 9: ttcacaaagcgccttcccccgtaaatga (mitochondrial DNA probe)
SEQ ID NO: 10: ccctgaactgcagatcaccaatgtggtag (nuclear DNA probe)
SEQ ID NO: 11: ttggatgcacaacatgaatcagg (Nix forward primer)
SEQ ID NO: 12: tcttctgactgagagctatggtc (Nix reverse primer)
SEQ ID NO: 13: gacgccttattcttctttgtc (GABARAP-L1 forward primer)
SEQ ID NO: 14: catgattgtcctcatacagttc (GABARAP-L1 reverse primer)
SEQ ID NO: 15: gtttgtggataagacagtcc (GABARAP-L2 DNA forward primer)
SEQ ID NO: 16: gaagccaaaagtgttctctc (GABARAP-L2 reverse primer)
SEQ ID NO: 17: ttccccttggccatcaaga (PINK1 forward primer)
SEQ ID NO: 18: accagctcctggctcattgt (PINK1 reverse primer)
SEQ ID NO: 19: gtcctctcccaagtccacac (β-actin forward primer)
SEQ ID NO: 20: gggagaccaaaagcttcat (β-actin reverse primer)
```

### DETAILED DESCRIPTION

### Definitions

As used herein, the terms “treatment” or “treating” mean: (1) improving or stabilizing the subject's condition or disease or (2) preventing or relieving the development or worsening of symptoms associated with the subject's condition or disease.

As used herein, the terms “prevent,” “preventing,” “prevention,” and the like refer to reducing the probability of developing a disorder or condition in a subject, who does not have, but is at risk of or susceptible to developing a disorder or condition.

As used herein, the terms “administration” or “administering” mean a route of administration for a compound disclosed herein. Exemplary routes of administration include, but are not limited to, oral, intravenous, intraperitoneal, intraarterial, and intramuscular. The preferred route of administration can vary depending on various factors, e.g., the components of the pharmaceutical composition comprising an agent as disclosed herein, site of the potential or actual disease and severity of disease.

As used herein, the terms “amount effective” or “effective amount” mean the amount of an agent disclosed herein that when administered to a subject for treating a disease, is sufficient to effect such treatment of the disease. Any improvement in the patient is considered sufficient to achieve treatment. An effective amount of an agent disclosed herein, used for the treatment of a neurodegenerative disease can vary depending upon the manner of administration, the age, body weight, and general health of the patient. Ultimately, the prescribers or researchers will decide the appropriate amount and dosage regimen.

As used herein, the terms “neurodegenerative disorder” and “neurodegenerative disease” are used interchangeably in this document and mean diseases of the nervous system (e.g., the central nervous system or peripheral nervous system) characterised by abnormal cell death. Examples of neurodegenerative conditions include Alzheimer disease, Down's syndrome, frontotemporal dementia, progressive supranuclear palsy, Pick's disease, Niemann-Pick disease, Parkinson's disease, Huntington's disease, dentatorubropallidoluysian atrophy, Kennedy's disease (also referred to as spinobulbar muscular atrophy), and spinocerebellar ataxia (e.g., type 1, type 2, type 3 (also referred to as Machado-Joseph disease), type 6, type 7, and type 17)), fragile X (Rett's) syndrome, fragile XE mental retardation, Friedreich's ataxia, myotonic dystrophy, spinocerebellar ataxia type 8, and spinocerebellar ataxia type 12, Alexander disease, Alper's disease, amyotrophic lateral sclerosis (or motor neuron disease), Hereditary spastic paraplegia, mitochondrial disease, ataxia telangiectasia, Batten disease (also referred to as Spielmeyer-Vogt-Sjogren-Batten disease), Canavan disease, Cockayne syndrome, corticobasal degeneration, Creutzfeldt-Jakob disease, ischemia stroke, Krabbe disease, Lewy body dementia, multiple sclerosis, multiple system atrophy, Pelizaeus-Merzbacher disease, Pick's disease, primary lateral sclerosis, Refsum's disease, Sandhoff disease, Schilder's disease, spinal cord injury, spinal muscular atrophy, Steele-Richardson-Olszewski disease, and Tabes dorsalis.

As used herein, the term “neurodegenerative disorders associated with mitochondrial dysfunction” means a neurodegenerative condition that is characterised by or implicated by mitochondrial dysfunction. Exemplary neurodegenerative conditions associated with mitochondrial dysfunction include, without limitation, Friedrich's ataxia, amyotrophic lateral sclerosis, mitochondrial myopathy, encephalopathy, lactacidosis, stroke (MELAS), myoclonic epilepsy with ragged red fibers (MERRF), Kearn-Sayre Syndrome, chronic progressive ophthalmoplegia, Alpers disease, Leigh's disease, epilepsy, Parkinson's disease, Alzheimer's disease, Huntington's disease and mitochondrial disease.

As used herein, the terms “subject” and “patient” are used herein interchangeably. They refer to a human or another mammal (e.g., mouse, rat, rabbit, dog, cat, cattle, swine, sheep, horse or primate) that can be afflicted with or is susceptible to a disease or disorder but may or may not have the disease or disorder. In certain embodiments, the subject is a human being.

As used herein, the term “agent” means any small molecule chemical compound, antibody, nucleic acid molecule, or polypeptide or fragment thereof.

As used herein, the term “mitophagy” refers to the process of removal of dysfunctional or damaged mitochondria from a cell. For example, mitophagy may occur by the process of autophagy characterised by the incorporation of the organelles into double membrane vesicles called autophagosomes, fusion of autophagosomes with lysosomes to form autophagolysosomes and subsequent degradation of the autophagolysosomes.

As used herein a “Nix polypeptide” means a protein or fragment thereof having at least 85%, at least 90%, at least 95%, at least 98%, at least 99% or 100% amino acid sequence identity to the amino acid sequence set out in SEQ ID NO: 1 and having Nix biological activity.

As used herein “Nix polynucleotide” means a nucleic acid molecule encoding a Nix polypeptide (e.g. SEQ ID NO: 2).

As used herein “Nix-mediated mitophagy” means autophagic clearance of mitochondria involving Nix and includes interaction of Nix with other proteins including proteins on the autophagosomal membrane such as LC3 and GABARAP-L1. Nix-mediated mitophagy can involve interaction of Nix with Parkin and can also occur independently of Parkin.

As used herein a “GABARAP-L1 polypeptide” means a protein or fragment thereof having at least 85%, at least 90%, at least 95%, at least 98%, at least 99% or 100% amino acid sequence identity to the amino acid sequence set out in SEQ ID NO: 3 and having GABARAP-L1 biological activity.

As used herein “GABARAP-L1 polynucleotide” means a nucleic acid molecule encoding a GABARAP-L1 polypeptide (e.g. SEQ ID NO: 4).

As used herein, the term “fragment” means a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000, 1500, 2000, 2500 or 3000 nucleotides or amino acids.

As used herein, the term “variant” when referring to a polypeptide means a polypeptide which contains a variation of the amino acid sequence of an original polypeptide which retains at least some of the biological activities of the original polypeptide or which may have an increased activity as compared to the original polypeptide.

As used herein the term “analog” refers to a molecule that is not identical but has analogous functional and/or structural features.

Where the terms “comprise”, “comprises”, “comprised” or “comprising” are used in this specification (including the claims) they are to be interpreted as specifying the presence of the stated features, integers, steps or components, but not precluding the presence of one or more other features, integers, steps or components, or group thereof.

A reference herein to a patent document or other matter which is given as prior art is not to be taken as an admission that that document or matter was known or that the information it contains was part of the common general knowledge as at the priority date of any of the claims.

### Mitophagy and Neurodegenerative Disorders

As discussed herein, mitochondria are essential organelles that regulate cellular energy metabolism and cell death. Accordingly, dysfunctional mitochondria and defects in their removal via mitophagy, has been linked to many pathophysiological disorders and diseases. For example, β-amyloid fragments have been demonstrated to target mitochondria and cause mitochondrial dysfunction in Alzheimer's disease and disruptions to mitochondrial function and physiology are brought about by the mutation to the single gene responsible for Huntington's disease. Accordingly, impaired mitophagy has been implicated in various neurodegenerative diseases such as Parkinson's disease and Alzheimer's disease.

On the contrary, the preservation or restoration of mitophagy is associated with neuroprotection. Indeed, it has recently been demonstrated that overexpression of PINK1 is associated with restoration of parkin-mediated mitophagy and neuroprotection in the context of Huntington's disease (Cell Death and Disease (2015) 6, e1617).

The present invention provides methods of preventing or treating neurodegenerative disorders in a subject through increasing Nix-mediated mitophagy in a cell.

### Mitophagy and Parkinson's Disease

Among the genes associated with monogenic Parkinson's disease (PD), mutations in parkin and PINK1 have been identified as the most common genetic cause of autosomal recessive early onset PD (EOPD). Parkin encodes E3 ubiquitin ligase and PINK1 encodes mitochondrial serine/threonine kinase, both of which are involved in the maintenance of healthy mitochondrial function and morphology. The role of mitochondrial dysfunction in PD has been understood from data demonstrating the exposure to mitochondrial toxins such as 1-methyl-4-phenyl-1,2,5,6-tetrahydropyridine (MPTP) and rotenone induced parkinsonism. Recent studies have demonstrated that Parkin is recruited to mitochondria in a PINK1 dependent manner upon the dissipation of mitochondrial membrane potential by carbonyl cyanide 3-chlorophenylhydrazone (CCCP) and thereby promotes ubiquitination and degradation of mitochondrial outer membrane proteins such as mitochondrial fusion proteins Mitofusin (Mfn) 1 and 2 via ubiquitin-proteasome system. This process prevents the fusion of dysfunctional mitochondria with a pool of healthy mitochondria and promotes clearance of dysfunctional mitochondria via autophagy-lysosomal system, a process referred to herein as mitophagy. Consistently, mutations in either parkin or PINK1 have been demonstrated to impair mitophagy in cellular models of PD, either using patient-derived cells or cells into which mutations in parkin or PINK1 have been introduced, leading to accumulation of dysfunctional mitochondria. Due to its detrimental effect on the mitochondrial quality control, impaired mitophagy is implicated in neurodegeneration and disease progression in PD.

Through the analysis of the phenotypic variability of parkinsonism observed in a family comprising various mutations in parkin (Koentjoro, et al., 2012, Mov Disord 27(10), 1299-303), the inventors have discovered that activation of an alternative mitophagy is capable of compensating for impaired Parkin-mediated mitophagy.

In cell models derived from a homozygous parkin mutation carrier (“Carrier cells”), who had no clinical manifestation of definite PD, normal mitochondrial function and clearance was observed. In contrast, the daughter of the carrier, or “proband”, who is a compound heterozygote and also lacks functional Parkin, presented with early onset PD. In cell models derived from the compound heterozygote (“Patient cells”), impairments to mitophagy were observed.

The inventors have surprisingly determined that expression of Nip3-like protein X (Nix) (also known as BNIP3L), and its binding partner y-aminobutyric acid type A receptor-associated protein like 1(GABARAP-L1), were elevated and associated with preserved mitophagy.

Nix is a mitochondrial outer membrane protein that has been demonstrated to play an important role in autophagic clearance of mitochondria. Consistent with its proposed function as a mitochondrial autophagic receptor, Nix has been shown to interact with proteins on the autophagosomal membrane such as LC3 and GABARAP-L1 and take part in mitochondrial translocation of Parkin and the induction of autophagy.

The inventors have demonstrated that activation of an alternative mitophagy involving Nix is able to preserve mitophagic function and is associated with the prevention of a neurodegenerative disorder in a subject.

Accordingly, the invention provides a method for the prevention or treatment of a neurodegenerative disorder in a subject, comprising administering to the subject a therapeutically effective amount of an agent that increases Nix-mediated mitophagy in a cell. In one embodiment the agent increases the biological activity or level of expression of a Nix polypeptide or fragment thereof, and/or a GABARAP-L1 polypeptide or fragment thereof in a cell.

In another embodiment, the agent is an expression vector encoding a Nix polypeptide or fragment thereof, and/or a GABARAP-L1 polypeptide or fragment thereof.

### Nix and GABARAP-L1 Polypeptides, Variants and Analogs

The invention provides for the use of Nix and/or GABARAP-L1 polypeptides or fragments or variants or analogs and expression vectors encoding Nix and/or GABARAP-L1 polypeptides or fragments or variants or analogs. In one embodiment, the invention provides methods for optimising a Nix and/or GABARAP-L1 amino acid sequence or nucleic acid sequence by producing an alteration in the sequence. Such alterations may include certain mutations, deletions, insertions, or post-translational modifications. In other embodiments, the invention further includes variants or analogs of any naturally occurring Nix and/or GABARAP-L1 polypeptide. Variants can differ from a naturally occurring polypeptide of the invention by amino acid sequence differences, by post-translational modifications, or by both. Variants of the Nix and/or GABARAP-L1 polypeptides will generally exhibit at least 85%, more preferably 90%, and most preferably 95% or even 99% identity with all or part of a naturally occurring amino acid sequence as described herein. The length of sequence comparison is at least 5, 10, 15 or 20 amino acid residues, preferably at least 25, 50, or 75 amino acid residues, and more preferably more than 100 amino acid residues.

Variants or analogs can differ from the naturally occurring polypeptides described herein by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Current Protocols in Molecular Biology” (Ausubel, 1987). Also included are cyclised peptides, molecules, and analogs which contain residues other than L-amino acids, e.g., D-amino acids or non-naturally occurring or synthetic amino acids, e.g., β or γ amino acids.

Amino acids include naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, for example, hydroxyproline, gamma-carboxyglutamate, and O-phosphoserine, phosphothreonine. An amino acid analog is a compound that has the same basic chemical structure as a naturally occurring amino acid, i.e., a carbon that is bound to a hydrogen, a carboxyl group, an amino group, and an R group (e.g., homoserine, norleucine, methionine sulfoxide, methionine methyl sulfonium), but that contains some alteration not found in a naturally occurring amino acid (e.g., a modified side chain); the term “amino acid mimetic” refers to chemical compounds that have a structure that is different from the general chemical structure of an amino acid, but that function in a manner similar to a naturally occurring amino acid. Amino acid analogs may have modified R groups (for example, norleucine) or modified peptide backbones, but retain the same basic chemical structure as a naturally occurring amino acid. In one embodiment, an amino acid analog is a D-amino acid, a β-amino acid, or an N-methyl amino acid.

Amino acids and analogs are well known in the art. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. In addition to full-length polypeptides, the invention also includes fragments of any one of the polypeptides of the invention. Non-protein Nix and/or GABARAP-L1 analogs having a chemical structure designed to mimic Nix and/or GABARAP-L1 functional activity can be administered according to methods of the invention. Nix and/or GABARAP-L1 variants and analogs may exceed the physiological activity of the original polypeptide. Methods of analog design are well known in the art, and synthesis of analogs can be carried out according to such methods by modifying the chemical structures such that the resultant analogs exhibit the activity of a reference Nix and/or GABARAP-L1 polypeptide. These chemical modifications include, but are not limited to, substituting alternative R groups and varying the degree of saturation at specific carbon atoms of a reference polypeptide. Preferably, the polypeptide analogs are relatively resistant to in vivo degradation, resulting in a more prolonged therapeutic effect upon administration. Assays for measuring functional activity include, but are not limited to, those described in the Examples below.

Accordingly, polynucleotide therapy featuring a polynucleotide encoding a Nix and/or GABARAP-L1 protein, variant, or fragment thereof is one therapeutic approach for treating or preventing a neurodegenerative disorder. Expression of such proteins in a cell comprising damaged or dysfunctional mitochondria is expected to promote the elimination of those mitochondria. Such nucleic acid molecules can be delivered to cells of a subject that has a neurodegenerative disorder or disease or is at risk of developing the same. The nucleic acid molecules must be delivered to the cells of a subject in a form in which they can be taken up so that therapeutically effective levels of a Nix and/or GABARAP-L1 protein or fragment thereof can be produced.

Expression vectors encoding Nix and/or GABARAP-L1 may be administered for global expression or may be used for the transduction of selected tissues. Transducing viral (e.g., retroviral, adenoviral, adeno-associated viral and lentiviral) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94: 10319, 1997). For example, a polynucleotide encoding a Nix and/or GABARAP-L1 protein, variant, or a fragment thereof, can be cloned into a retroviral vector and expression can be driven from its endogenous promoter, from the retroviral long terminal repeat, or from a promoter specific for a target cell type of interest. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244: 1275-1281, 1989; Eglitis et al., BioTechniques 6:608-614, 1988; Tolstoshev et al., Current Opinion in Biotechnology 1 :55-61, 1990; Sharp, The Lancet 337 : 1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346). Preferably, a viral vector is used to administer a Nix and/or GABARAP-L1 polynucleotide systemically.

Non-viral approaches can also be employed for the introduction of therapeutic to a cell of a patient requiring treatment or prevention of a neurodegenerative disease. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101 :512, 1983), asialoorosomucoid-polylysine conjugation (Wu et al., Journal of Biological Chemistry 263: 14621, 1988; Wu et al., Journal of Biological Chemistry 264:16985, 1989), or by micro-injection under surgical conditions (Wolff et al., Science 247:1465, 1990). Preferably the nucleic acids are administered in combination with a liposome and protamine.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. Transplantation of normal genes into the affected tissues of a patient can also be accomplished by transferring a normal nucleic acid into a cultivatable cell type ex vivo (e.g., an autologous or heterologous primary cell or progeny thereof), after which the cell (or its descendants) are injected into a targeted tissue.

cDNA expression for use in polynucleotide therapy methods can be directed from any suitable expression system (e.g. lentiviral expression system) using any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types can be used to direct the expression of a nucleic acid. The enhancers used can include, without limitation, those that are characterised as tissue- or cell-specific enhancers. Alternatively, if a genomic clone is used as a therapeutic construct, regulation can be mediated by the cognate regulatory sequences or, if desired, by regulatory sequences derived from a heterologous source.

Another therapeutic approach included in the invention involves administration of a recombinant therapeutic, such as a recombinant Nix and/or GABARAP-L1 protein, variant, or fragment thereof, either directly to the site of a potential or actual disease-affected tissue or systemically (for example, by any conventional recombinant protein administration technique). The dosage of the administered protein depends on a number of factors, including the size and health of the individual patient. For any particular subject, the specific dosage regimes should be adjusted over time according to the individual need and the professional judgment of the person administering or supervising the administration of the compositions.

### Screens for Agents that Increase Nix-Mediated Mitophagy

As discussed herein, impairment to Parkin-related mitophagy as a result of mutations to parkin or PINK1 may be compensated for by an increase in Nix-mediated mitophagy. Given that subjects having mitochondrial defects have a mixed population of healthy and defective mitochondria, agents that selectively reduce the number of defective mitochondria are useful for the treatment of neurodegenerative disorders. If desired, agents that increase the expression or biological activity of Nix and/or GABARAP-L1 are tested for efficacy in enhancing the selective elimination of defective mitochondria in a cell (e.g., a cell comprising a genetic defect in mtDNA, a cell comprising a genetic mutation in parkin or PINK1, a cell of the substantia nigra or a dopaminergic neuronal cell). Such methods are particularly useful for personalised medicine applications, for example, in identifying agents that are likely to be beneficial for a subject having a neurodegenerative disorder. In one example, a candidate compound is added to the culture medium of cells (e.g., neuronal cultures) prior to, concurrent with, or following the addition of a mitochondrial uncoupling agent or other agent that induces mitophagy. Mitochondrial function and the degree of mitophagy in the cells is then measured using standard methods known to the skilled addressee, including those described herein. The mitochondrial function and/or degree of mitophagy in the presence of the candidate agent are compared to the level measured in a corresponding control culture that did not receive the candidate agent.

In one embodiment, the agent's ability to promote the selective elimination of defective mitochondria is assayed in a cell comprising a mutation in parkin and/or PINK1. A compound that promotes an increase in Nix and/or GABARAP-L1 expression or biological activity, or a reduction in defective mitochondria is identified as useful in the invention; such a candidate compound may be used, for example, as a therapeutic to prevent, delay, ameliorate, stabilise, or treat a neurodegenerative disorder characterised by mitochondrial dysfunction.

In one embodiment, the present invention provides a method for identifying an agent useful for the prevention or treatment of a neurodegenerative disorder in a subject comprising: (a) contacting a cell with an agent; and (b) detecting an increase in the biological activity or expression of a polypeptide associated with Nix-mediated mitophagy in the cell relative to a control cell not contacted with the agent, or (c) detecting an increase in the expression of a polynucleotide encoding a polypeptide associated with Nix-mediated mitophagy in the cell relative to a control cell not contacted with the agent, wherein an agent that increases said activity or said expression is identified as useful for the treatment of a neurodegenerative disorder.

In another embodiment, the present invention provides a method for identifying an agent useful for the prevention or treatment of a neurodegenerative disorder in a subject comprising: (a) contacting a cell with an agent; and (b) detecting an increase in the biological activity or expression of a Nix polypeptide and/or GABARAP-L1 polypeptide in the cell relative to a control cell not contacted with the agent, or (c) detecting an increase in the expression of a polynucleotide encoding a Nix polypeptide and/or GABARAP-L1 polypeptide in the cell relative to a control cell not contacted with the agent, wherein an agent that increases said activity or said expression is identified as useful for the treatment of a neurodegenerative disorder.

An agent isolated by this method (or any other appropriate method) may, if desired, be further purified (e.g., by high performance liquid chromatography). In addition, such candidate agents may be tested for their ability to modulate mitophagy in a neuronal cell. In other embodiments, the agent's activity is measured by identifying an increase in mitochondrial function, a reduction in cell death, or an increase in cell survival. Agents isolated by this approach may be used, for example, as therapeutics to treat a neurodegenerative disorder associated with mitochondrial dysfunction in a subject.

One skilled in the art appreciates that the effects of a candidate compound on a cell comprising defective mitophagy is typically compared to a corresponding control cell in the absence of the candidate compound.

Candidate agents include small molecules, peptides, peptide mimetics, polypeptides, and nucleic acid molecules. Each of the sequences listed herein may also be used in the discovery and development of a therapeutic compound for the treatment of a neurodegenerative disorder. The encoded protein, upon expression, can be used as a target for the screening of drugs. Additionally, the DNA sequences encoding the amino terminal regions of the encoded protein or Shine-Delgarno or other translation facilitating sequences of the respective mRNA can be used to construct sequences that promote the expression of the coding sequence of interest. Such sequences may be isolated by standard techniques (Ausubel et al., supra). Small molecules of the invention preferably have a molecular weight below 2,000 daltons, more preferably between 300 and 1,000 daltons, and most preferably between 400 and 700 daltons. It is preferred that these small molecules are organic molecules.

The invention also includes novel agents identified by the above-described screening assays. Optionally, such agents are characterised in one or more appropriate animal models to determine the efficacy of the compound for the treatment of a neurodegenerative disorder. Desirably, characterisation in an animal model can also be used to determine the toxicity, side effects, or mechanism of action of treatment with such a compound. Furthermore, a novel agent identified in any of the above-described screening assays may be used for the treatment of a neurodegenerative disorder in a subject. Such agents are useful alone or in combination with other conventional therapies known in the art.

### Cells for Use in Screens

In one embodiment, the screens described herein are carried out in cells comprising a mutation in parkin or PINK1.

In another embodiment, the screens described herein are carried out in dopaminergic cells having neuronal characteristics. Such cells are known in the art and include, for example, BE(2)-M17 neuroblastoma cells (Martin et al., J Neurochem. 2003 November; 87(3):620-30), Cath.a-differentiated (CAD) cells (Arboleda et al., J Mol Neurosci. 2005; 27(1):65-78), CSM14.1 (Haas et al., J Anat. 2002 July; 201(1):61-9), MN9D (Chen et al., Neurobiol Dis. 2005 August; 19(3):419-26), N27 cells (Kaul et al., J Biol Chem. 2005 Aug. 5; 280(31):28721-30), PC12 (Gorman et al., Biochem Biophys Res Commun. 2005 Feb. 18; 327(3):801-10), SN4741 (Nair et al., Biochem J. 2003 Jul. 1; 373(Pt 1):25-32), CHP-212, SH-SYSY, and SK-N-BE. In an alternative embodiment the screens described herein may be carried out in dopaminergic cells derived from a stem cell, an iPS cell, or a progenitor cell.

In another embodiment the screens described herein may be carried out in neurons, fibroblasts, olfactory neurospheres, or neuroprogenitors or neurons derived from fibroblasts or olfactory neurospheres or neuroprogenitors.

### Test Agents and Extracts

In general, agents capable of modulating mitophagy are identified from large libraries of both natural product or synthetic (or semi-synthetic) extracts or chemical libraries or from polypeptide or nucleic acid libraries, according to methods known in the art. Those skilled in the field of drug discovery and development will understand that the precise source of test extracts or agent is not critical to the screening procedure(s) of the invention. Agents used in screens may include known agents (for example, known therapeutics used for other diseases or disorders). Alternatively, virtually any number of unknown chemical extracts or agent can be screened using the methods described herein. Examples of such extracts or agents include, but are not limited to, plant-, fungal-, prokaryotic- or animal-based extracts, fermentation broths, and synthetic agents, as well as modification of existing agents.

Numerous methods are also available for generating random or directed synthesis (e.g., semi-synthesis or total synthesis) of any number of chemical agents, including, but not limited to, saccharide-, lipid-, peptide-, and nucleic acid-based agent. Synthetic compound libraries are commercially available from Brandon Associates (Merrimack, N.H.) and Aldrich Chemical (Milwaukee, Wis.). Alternatively, a chemical agent to be used as candidate agent can be synthesised from readily available starting materials using standard synthetic techniques and methodologies known to those of ordinary skill in the art. Synthetic chemistry transformations and protecting group methodologies (protection and deprotection) useful in synthesizing the agent identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Alternatively, libraries of natural agents in the form of bacterial, fungal, plant, and animal extracts are commercially available from a number of sources, including Biotics (Sussex, UK), Xenova (Slough, UK), Harbor Branch Oceanographic Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. U.S.A. 91: 11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37: 1233, 1994. Furthermore, if desired, any library or compound is readily modified using standard chemical, physical, or biochemical methods.

Libraries of agents may be presented in solution (e.g., Houghten, Biotechniques 13:412-421. 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc. Natl. Acad. Sci. U.S.A. 89: 1865-1869, 1992) or on phage (Scott and Smith, Science 249:386-390, 1990; Devlin, Science 249:404-406, 1990; Cwirla et al., Proc. Natl. Acad. Sci. U.S.A. 87:6378-6382, 1990; Felici, J. Mol. Biol. 222:301-310, 1991; Ladner supra.).

In addition, those skilled in the art of drug discovery and development readily understand the methods for dereplication (e.g., taxonomic dereplication, biological dereplication, and chemical dereplication, or any combination thereof) or the elimination of replicates or repeats of materials already known for their activity should be employed whenever possible.

When a crude extract of interest is identified, further fractionation of the positive lead extract is necessary to isolate chemical constituents responsible for the observed effect. Thus, the goal of the extraction, fractionation, and purification process is the careful characterisation and identification of a chemical entity within the crude extract that alters the transcriptional activity of a gene encoding a polypeptide associated with Nix-mediated mitophagy. Methods of fractionation and purification of such heterogeneous extracts are known in the art. If desired, agents shown to be useful as therapeutics for the treatment of a neurodegenerative disorder are chemically modified according to methods known in the art.

### Pharmaceutical Therapeutics

The invention provides agents that increase the expression or activity of Nix and/or GABARAP-L1, including agents identified in the above-identified screens, for the treatment of a neurodegenerative disorder. In one embodiment, the invention provides pharmaceutical compositions comprising an expression vector encoding a Nix and/or GABARAP-L1 polypeptide. In another embodiment, a chemical entity discovered to have medicinal value using the methods described herein is useful as a drug or as information for structural modification of existing agent, e.g., by rational drug design. For therapeutic uses, the compositions or agents identified using the methods disclosed herein may be administered systemically, for example, formulated in a pharmaceutically-acceptable carrier. Preferable routes of administration include, for example, subcutaneous, intravenous, intraperitoneal, intramuscular, or intradermal injections, intranasal (e.g. nasal spray) or transdermal (e.g. topical patch) administration, that provide continuous, sustained levels of the drug in the patient. Treatment of human patients or other animals will be carried out using a therapeutically effective amount of a neurodegenerative disorder therapeutic in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and the clinical symptoms of the neurodegenerative disorder. Generally, amounts will be in the range of those used for other agents used in the treatment of mitochondrial disease, although in certain instances lower amounts will be needed because of the increased specificity of the compound. A compound is administered at a dosage that controls the clinical or physiological symptoms of a neurodegenerative disorder as determined by a diagnostic method known to one skilled in the art, or using any assay that measures the transcriptional regulation of a gene associated with a neurodegenerative disorder, or associated with Nix-mediated mitophagy (e.g., Nix).

### Formulation of Pharmaceutical Compositions

The administration of an agent of the invention or analog thereof for the treatment of a neurodegenerative disorder may be by any suitable means that results in a concentration of the therapeutic that, combined with other components, is effective in ameliorating, reducing, or stabilising the neurodegenerative disorder or a symptom thereof. In one embodiment, administration of the agent reduces the percentage of dysfunctional or defective mitochondria in a cell and/or increases the percentage of healthy mitochondria.

Methods of administering such agents are known in the art. The invention provides for the therapeutic administration of an agent by any means known in the art. The compound may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York). Suitable formulations include forms for oral administration, depot formulations, formulations for delivery by a patch, semisolid dosage forms to be topically, transnasally or transdermally delivered.

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimisation of undesirable side effects associated with fluctuations in the plasma level of the active substance (sawtooth kinetic pattern); (iv) formulations that localise action by, e.g., spatial placement of a controlled release composition adjacent to or in the central nervous system or cerebrospinal fluid; (v) formulations that allow for convenient dosing, such that doses are administered, for example, once every one or two weeks; and (vi) formulations that target a neurodegenerative disorder by using carriers or chemical derivatives to deliver the therapeutic agent to a particular cell type (e.g., a neuronal cell at risk of cell death) whose function is perturbed in the neurodegenerative disorder. For some applications, controlled release formulations obviate the need for frequent dosing during the day to sustain the plasma level at a therapeutic level. Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

### Parenteral Compositions

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active therapeutic(s), the composition may include suitable parenterally acceptable carriers and/or excipients. The active therapeutic(s) may be incorporated into microspheres, microcapsules, nanoparticles, liposomes, or the like for controlled release. Furthermore, the composition may include suspending, solubilising, stabilising, pH-adjusting agents, tonicity adjusting agents, and/or dispersing, agents.

As indicated above, the pharmaceutical compositions according to the invention may be in the form suitable for sterile injection. To prepare such a composition, the suitable active therapeutic(s) are dissolved or suspended in a parenterally acceptable liquid vehicle.

### Controlled Release Parenteral Compositions

Controlled release parenteral compositions may be in the form of suspensions, microspheres, microcapsules, magnetic microspheres, oil solutions, oil suspensions, or emulsions. Alternatively, the active drug may be incorporated in biocompatible carriers, liposomes, nanoparticles, implants, or infusion devices. Materials for use in the preparation of microspheres and/or microcapsules are, e.g., biodegradable bioerodible polymers such as polygalactia poly-(isobutyl cyanoacrylate), poly(2-hydroxyethyl-L-glutam-nine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

### Solid Dosage Forms for Oral Use

Formulations for oral use include tablets containing an active ingredient(s) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinised starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticisers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug in a predetermined pattern (e.g., in order to achieve a controlled release formulation) or it may be adapted not to release the active drug until after passage of the stomach (enteric coating). The coating may be a sugar coating, a film coating (e.g., based on hydroxypropyl methylcellulose, methylcellulose, methylhydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose).

Furthermore, a time delay material such as, e.g., glyceryl monostearate or glyceryl distearate may be employed.

The solid tablet compositions may include a coating adapted to protect the composition from unwanted chemical changes, (e.g., chemical degradation prior to the release of the active neurodegenerative disorder therapeutic substance). The coating may be applied on the solid dosage form in a similar manner as that described in Encyclopedia of Pharmaceutical Technology, supra.

At least two active neurodegenerative disorder therapeutics may be mixed together in the tablet, or may be partitioned. In one example, the first active therapeutic is contained on the inside of the tablet, and the second active therapeutic is on the outside, such that a substantial portion of the second active therapeutic is released prior to the release of the first active therapeutic.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment.

### Controlled Release Oral Dosage Forms

Controlled release compositions for oral use may be constructed to release the active neurodegenerative disorder therapeutic by controlling the dissolution and/or the diffusion of the active substance. Dissolution or diffusion controlled release can be achieved by appropriate coating of a tablet, capsule, pellet, or granulate formulation of agent, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A controlled release composition containing one or more therapeutic agents may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the compound(s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

### Topical Administration Forms

Dosage forms for the semisolid topical administration of an agent of this invention include ointments, pastes, creams, lotions, and gels. The dosage forms may be formulated with mucoadhesive polymers for sustained release of active ingredients at the area of application to the skin. The active compound may be mixed under sterile conditions with a pharmaceutically acceptable carrier, and with any preservatives, buffers, or propellants, which may be required. Such topical preparations can be prepared by combining the compound of interest with conventional pharmaceutical diluents and carriers commonly used in topical liquid, cream, and gel formulations.

Ointments and creams may, for example, be formulated with an aqueous or oily base with the addition of suitable thickening and/or gelling agents. Such bases may include water and/or an oil (e.g., liquid paraffin, vegetable oil, such as peanut oil or castor oil). Thickening agents that may be used according to the nature of the base include soft paraffin, aluminum stearate, cetostearyl alcohol, propylene glycol, polyethylene glycols, woolfat, hydrogenated lanolin, beeswax, and the like.

Lotions may be formulated with an aqueous or oily base and, in general, also include one or more of the following: stabilizing agents, emulsifying agents, dispersing agents, suspending agents, thickening agents, coloring agents, perfumes, and the like. The ointments, pastes, creams and gels also may contain excipients, including, but not limited to, animal and vegetable fats, oils, waxes, paraffins, starch, tragacanth, cellulose derivatives, polyethylene glycols, silicones, bentonites, silicic acid, talc and zinc oxide, or mixtures thereof.

Suitable excipients, depending on the agent, include petrolatum, lanolin, methylcellulose, sodium carboxymethylcellulose, hydroxpropylcellulose, sodium alginate, carbomers, glycerin, glycols, oils, glycerol, benzoates, parabens and surfactants. It will be apparent to those of skill in the art that the solubility of a particular compound will, in part, determine how the compound is formulated. An aqueous gel formulation is suitable for water soluble agent. Where a compound is insoluble in water at the concentrations required for activity, a cream or ointment preparation will typically be preferable. In this case, oil phase, aqueous/organic phase and surfactant may be required to prepare the formulations. Thus, based on the solubility and excipient-active interaction information, the dosage forms can be designed and excipients can be chosen to formulate the prototype preparations.

The topical pharmaceutical compositions can also include one or more preservatives or bacteriostatic agents, e.g., methyl hydroxybenzoate, propyl hydroxybenzoate, chlorocresol, benzalkonium chlorides, and the like. The topical pharmaceutical compositions also can contain other active ingredients including, but not limited to, antimicrobial agents, particularly antibiotics, anesthetics, analgesics, and antipruritic agents.

### Dosage

Human dosage amounts can initially be determined by extrapolating from the amount of compound used in mice, as a skilled artisan recognises it is routine in the art to modify the dosage for humans compared to animal models. In certain embodiments it is envisioned that the dosage may vary from between about 1 mg compound/Kg body weight to about 5000 mg compound/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other embodiments this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, 5000 mg/Kg body weight. In other embodiments, it is envisaged that higher does may be used, such doses may be in the range of about 5 mg compound/Kg body to about 20 mg compound/Kg body. In other embodiments the doses may be about 8, 10, 12, 14, 16 or 18 mg/Kg body weight. Of course, this dosage amount may be adjusted upward or downward, as is routinely done in such treatment protocols, depending on the results of the initial clinical trials and the needs of a particular patient.

### Therapeutic Methods

The present invention provides methods of treating a neurodegenerative disorder or symptoms thereof by modulating the elimination of dysfunctional or defective mitochondria via Nix-mediated mitophagy. The methods comprise administering a therapeutically effective amount of a pharmaceutical composition comprising an agent that modulates the clearance of dysfunctional or defective mitochondria from a cell using the methods described herein to a subject (e.g., a mammal such as a human). Thus, one embodiment is a method of treating a subject suffering from or susceptible to a neurodegenerative disorder. The method includes the step of administering to the subject a therapeutically effective amount of an agent herein described sufficient to treat the disorder, under conditions such that the disorder is treated.

The methods herein include administering to the subject (including a subject identified as in need of such treatment) an effective amount of an agent described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention, which include prophylactic treatment, in general comprise administration of a therapeutically effective amount of the agent herein, to a subject (e.g., animal, human) in need thereof, including a mammal, particularly a human. Such treatment will be suitably administered to subjects, particularly humans, suffering from, having, susceptible to, or at risk of developing a neurodegenerative disorder.

Determination of those subjects “at risk” can be made by any objective or subjective determination by a diagnostic test or opinion of a subject or health care provider (e.g., genetic test, enzyme or protein marker, family history, and the like).

In one embodiment, the invention provides a method of monitoring treatment progress. The method includes the step of determining a level of Nix and/or GABARAP-L1 expression or other diagnostic measurement (e.g., screen, assay) in a subject suffering from or at risk of developing a neurodegenerative disorder, in which the subject has been administered a therapeutic amount of an agent as herein described, sufficient to treat the disorder or symptoms thereof. The level of expression determined in the method can be compared to known levels of expression in either healthy normal controls or in other afflicted patients to establish the subject's disease status. In preferred embodiments, a second level of expression in the subject is determined at a time point later than the determination of the first level, and the two levels are compared to monitor the course of disease or the efficacy of the therapy. In certain preferred embodiments, a pre-treatment level of expression in the subject is determined prior to beginning treatment according to this invention; this pre-treatment level of Marker can then be compared to the level of Marker in the subject after the treatment commences, to determine the efficacy of the treatment. The following examples are provided to illustrate the invention, not to limit it. Those skilled in the art will understand that the specific constructions provided below may be changed in numerous ways, consistent with the above described invention while retaining the critical properties of the agent or combinations thereof.

### Kits

The invention provides kits for the treatment or prevention of a neurodegenerative disorder. In one embodiment, the kit includes a therapeutic or prophylactic composition containing an effective amount of an agent of the invention (e.g., an agent which increases Nix-mediated mitophagy in a cell, including agents which increase the expression and/or activity of Nix; a Nix polypeptide or fragment or variant thereof, and/or a GABARAP-L1 polypeptide or fragment or variant thereof, or expression vectors encoding the same) in unit dosage form. In some embodiments, the kit comprises a sterile container which contains a therapeutic or prophylactic compound; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired an agent of the invention is provided together with instructions for administering it to a subject having or at risk of developing a neurodegenerative disorder. The instructions will generally include information about the use of the composition for the treatment or prevention of the neurodegenerative disorder. In other embodiments, the instructions include at least one of the following: description of the compound; dosage schedule and administration for treatment or prevention of a neurodegenerative disorder or symptoms thereof precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### Combination Therapies

Optionally, an agent having therapeutic or prophylactic efficacy may be administered in combination with any other standard therapy for the treatment of a neurodegenerative disorder. If desired, agents of the invention may be administered alone or in combination with a conventional therapeutic useful for the treatment of a neurodegenerative disorder. For example, therapeutics useful for the treatment of Parkinson's disease include, but are not limited to, deprenyl, amantadine or anticholinergic medications, levodopa, carbidopa, entacapone, pramipexole, rasagiline, antihistamines, antidepressants, dopamine agonists, monoamine oxidase inhibitors (MAOIs), and others.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature (such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991)). These techniques are applicable to the production of the polynucleotides and polypeptides of the invention, and, as such, may be considered in making and practicing the invention.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1. Mitochondrial Function is Normal in the Parkin-Deficient Carrier Cells

The inventors identified a healthy homozygous parkin mutation carrier who had no functional Parkin protein. As mitochondria are the main cellular organelle generating energy in the form of ATP and the primary target affected by mutations in parkin, the mitochondrial ATP synthesis rate in fibroblasts from the Parkin-deficient mutation carrier (“carrier” hereafter) and the patient (compound heterozygote lacking functional Parkin; hereinafter “patient”) were determined.

### Methods

### Cell Culture

The protocols for establishment and culture of human fibroblasts and human olfactory neurosphere cell lines have previously been described (Koentjoro, et al., 2012, Mov Disord 27(10), 1299-303; Park, et al., 2011; Hum Mutat 32(8), 956-64). Cells were subcultured to a maximum of 15 passages for all experiments.

### Assessment of ATP Synthesis Rate

ATP synthesis rate was determined as previously described (Shepherd, et al., 2006). Briefly, fibroblasts were harvested by trypsinisation followed by determining the total protein concentration using BCA protein assay kit (Thermo Scientific, Rockford, IL, USA) according to the manufacturer's instructions. Cells were diluted in a cell suspension buffer (150 mM KCl, 25 mM Tris-HCl pH 7.6, 2 mM EDTA pH 7.4, 10 mM KPO₄ pH 7.4, 0.1 mM MgCl₂ and 0.1% (w/v) BSA (Sigma)) at 1 mg/mL total protein. ATP synthesis was induced by incubation of 250 μL of the cell suspension with 750 μL of a substrate buffer (10 mM malate, 10 mM pyruvate, 1 mM ADP, 40 μg/mL digitonin and 0.15 mM adenosine pentaphosphate (Sigma)) for 10 minutes at 37° C. Following this incubation, the reaction was stopped by addition of 450 μL of boiling quenching buffer (100 mM Tris-HCl, 4 mM EDTA pH 7.75 (Sigma)) to 50 μL aliquot of the reaction mixture and incubating for 2 minutes at 100° C. The resulting reaction mixture was further diluted 1:10 in quenching buffer and the amount of ATP was measured in an FB10 luminometer (Berthold Detection Systems, Pforzheim, Germany) with the ATP Bioluminescence Assay Kit (Roche Diagnostics, Basel, Switzerland), according to the manufacturer's instructions.

### Cytotoxicity Test

Cell death was determined using CytoTox 96 Non-Radioactive Cytotoxicity Assay Kits (Promega, Madison, WI, U.S.A.) according to the manufacturer's protocol. In brief, human olfactory neurosphere cells were seeded in a 24-well culture plate at 50,000 cells per well and cultured for 48 hours. Cells were then exposed to increasing doses of rotenone (Sigma; 1.5, 2.5 and 12.5 μM) for 72 hours. A 50 μL aliquot of culture media was incubated with a substrate mix for 30 minutes. Lactate dehydrogenase (LDH) activity was measured spectrophotometrically at 490 nm using a Benchmark Microplate Reader (Bio-Rad, Hercules, CA, U.S.A.).

### Results

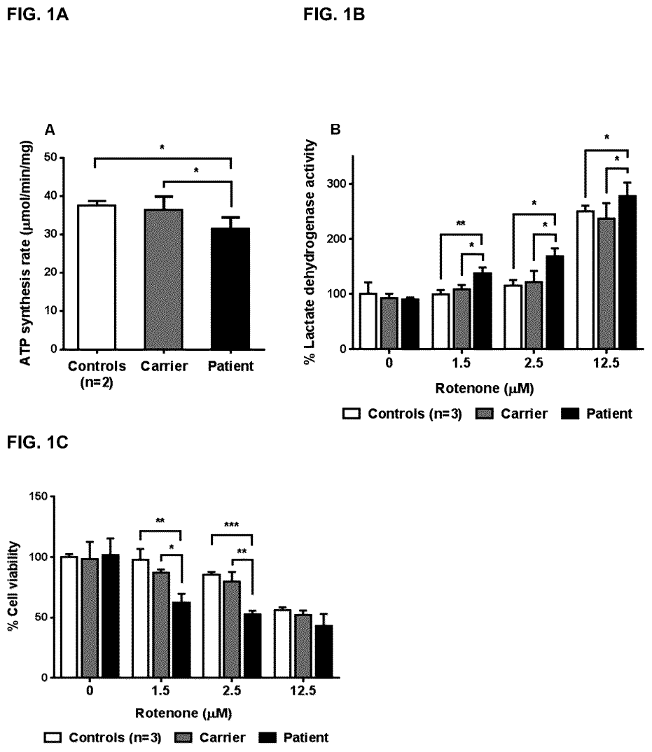

When compared to controls (37.4±1.2), the ATP synthesis rate was significantly reduced in the patient cells (31.4±3.0, p<0.05), but not in the carrier cells (36.4±3.5) (FIG. 1A). Cells were then treated with rotenone to examine its effect in the carrier cells. Rotenone is a mitochondrial complex I inhibitor and has been shown to increase cytotoxicity in the cells with mitochondrial dysfunction such as Parkin-related PD cell model. Upon exposure to rotenone, the patient cells displayed an increased toxicity while the control and the carrier cells responded similarly (FIGS. 1B and 1C).

FIG. 1 shows (A) adenosine triphosphate (ATP) synthesis rate was normal in the fibroblast derived from the carrier, but significantly reduced in the patient fibroblast, when compared to controls. (B) Human olfactory neurosphere cells were tested for rotenone sensitivity using lactate dehydrogenase (LDH) activity released into the media. Increasing doses of rotenone (0, 1.5, 2.5 and 12.5 μM) significantly elevated the LDH activity in the media of patient cells (black bars) in a dose-response manner, when compared to the controls (white bars) while the carrier (grey bars) showed a similar degree of cytotoxicity to the control cells. (C) Rotenone-induced cell death in human olfactory neurosphere cells. Cells were tested for rotenone sensitivity using the cytotoxicity assay described above. Increasing doses of rotenone (0, 1.5, 2.5 and 12.5 μM) significantly reduced cell viability in the patient cells (black bars) in a dose-response manner, when compared to the controls (white bars) and the carrier cells (grey bars). *; p<0.05, **; p<0.01 and ***; p<0.001 in one-way ANOVA followed by post hoc Tukey's HSD multiple comparison test.

These results indicate mitochondrial dysfunction in the patient but preserved mitochondrial function in the carrier cells despite the same condition of Parkin deficiency.

### Example 2. CCCP-Induced Mitophagy is Normal in the Carrier Cells

The normal mitochondrial function observed in the carrier cells suggested normal function of mitochondrial quality control compensating for the loss of Parkin. Mitophagy was induced in the carrier cells using CCCP and examined using a combination of three different methods; measurement of the mitochondrial mass by citrate synthase activity (a mitochondrial matrix enzyme), the mtDNA content by quantitative real-time PCR and the co-localisation of mitochondria and autophagosomes by confocal microscopy. To visually monitor mitophagy using confocal microscopy, fibroblasts expressing GFP-LC3 for autophagosomal marker and RFP-Mito for mitochondrial marker were generated.

### Methods

### Lentivirus Production and Establishment of Cell Lines

The green fluorescent protein (GFP)-tagged LC3 vector was a kind gift from Dr Ernst Wolvetang. Lentivirus for the expression of GFP-LC3 was produced using the Lenti-X Lentiviral HTX Packaging system (Clontech, Mountain View, CA, USA) and Lipofectamine 2000 (Invitrogen, Carlsbad, CA, USA) according to the manufacturer's instruction. The media containing lentivirus was collected at 48 and 72 hours post-transfection followed by concentration using the Lenti-X concentrator (Clontech) before measurement of viral titre.

For generation of stable cell lines expressing GFP-LC3, fibroblasts were transduced with 1 multiplicity of infection (MOI) lentivirus in the presence of 4 μg/mL polybrene (Sigma, St. Louis, MO, USA) for 24 hours and subsequently grown in culture media containing 2 μg/mL blasticidin (Invitrogen) for selection.

### Assessment of Mitochondrial Clearance

Fibroblasts were seeded in a 6-well culture plate at 200,000 cells per well and treated with either DMSO or CCCP (Sigma) at 10 μM for 24 hours to induce mitophagy by reducing the mitochondrial membrane potential.

For measurement of mitochondrial mass, activity of citrate synthase (a mitochondrial matrix enzyme) was determined using Citrate Synthase Assay Kit (Sigma) according to the manufacturer's instructions. Briefly, fibroblasts were harvested with a cell scraper and resuspended in a cell lysis buffer (CelLytic M Cell Lysis Reagent supplemented with a cocktail of protease inhibitors (Sigma)), followed by brief sonication. After determining the protein concentration using a BCA Protein Assay Kit (Thermo Scientific) according to the manufacturer's instructions, 10 μg of total protein was mixed with a substrate buffer (1×assay buffer, 300 μM acetyl CoA, 100 μM 5,5′-Dithiobis-(2-nitrobenzoic acid)), followed by the addition of 100 μM oxaloacetate solution to start the reaction. Optical absorbance of the reaction mixture at 412 nm (OD412) was taken every 10 seconds for 1.5 minutes before and after the addition of the oxaloacetate solution. Citrate synthase activity was determined by subtracting the OD412 per minute before addition of oxaloacetate (the basal activity) from OD412 per minute after addition of oxaloacetate.

Quantification of mitochondrial DNA was carried out using real time quantitative polymerase chain reaction (qPCR) as previously reported (Parfait, et al., 1998). In brief, fibroblasts were harvested with a cell scraper. Total DNA (i.e. nuclear DNA [nDNA] and mitochondrial DNA [mtDNA]) was then extracted using QIAamp DNA Mini Kit (Qiagen, Hilden, Germany) according to the manufacturer's instructions. A multiplex qPCR analysis was performed using TaqMan Gene Expression Master Mix (Invitrogen) on the Rotor Gene 6000 (Qiagen) according to the manufacturer's instructions. The primers and TaqMan probes used in the reaction are listed in Table 1 below. The amount of mtDNA was calculated relative to the nDNA using the Rotor-Gene 6000 Series Software v.1.7.

TABLE 1
Primers and Probes for Quantification of mitochondrial DNA

| Primers | Forward (5′-3′) | Reverse (5′-3′) |
|---|---|---|
| mtDNA | AGGAGAAGAGAAATAAGGCC | TAAGAAGAGGAATTGAACCTCTGACTGTAA |
| nDNA | TTTTTGTGTGCTCTCCCAGGTCT | TGGTCACTGGTTGGTTGGC |
| Probes | Sequence (5′-3′) | |
| mtDNA | VIC-TTCACAAAGCGCCTTCCCCCGTAAATGA-TAMRA | |
| nDNA | FAM-CCCTGAACTGCAGATCACCAATGTGGTAG-TAMRA | |

Co-localisation study of mitochondria and autophagosomes was performed using confocal microscopy. Briefly, 30,000 fibroblasts expressing GFP-LC3 were seeded on to 35 mm μ-Dishes (Ibidi, Martinsried, Germany) and cultured for 48 hours, followed by transduction with the CellLight Mitochondria-RFP BacMan 2.0 (Invitrogen) to label mitochondria according to the manufacturer's instructions. Following 24 hours incubation, the cells were treated with 20 μM CCCP for 4 hours (Sigma) to induce mitophagy and subjected to live cell imaging using a Leica TCS SP5 II confocal microscope (Leica Microsystems, Wetzlar, Germany). Images of fifty individual cells from at least two independent experiments were taken and analysed. The degree of co-localisation was determined using the LAS-AF software v.2.6.0 (Leica Microsystems) with following conditions and calculations: degree of co-localisation [%]=area co-localisation÷area foreground with threshold and background subtraction set at 30%; area foreground=area image−area background.

### Results

Exposure to CCCP caused a significant reduction of mitochondrial mass in the controls (an average of 51.8% reduction compared to the vehicle control, p<0.001) and the carrier cells (38.7% reduction compared to the vehicle control, p<0.001), but not in the patient cells (p=0.16). Similarly, mtDNA content was significantly decreased by CCCP treatment in the controls (35.4%, p<0.01) and the carrier cells (27.6%, p<0.01), but not in the patient cells (p=0.84). In confocal microscopy, a minimal co-localisation between GFP-LC3 and RFP-Mito was observed prior to CCCP treatment (data not shown). Upon induction of mitophagy by CCCP, a marked increase of co-localisation between GFP-LC3 and RFP-Mito was observed in the control and the carrier cells, suggesting an elevated mitophagy, whereas such change was not observed in the patient cells. The quantification of co-localisation revealed a significantly lower degree of co-localisation in the patient cells (8.6±9.5%, p<0.01) compared to the control (100±28.3%), suggesting a defective mitophagy, whereas the co-localisation observed in the carrier cells (101.9±36.5%) was comparable to the control cells.

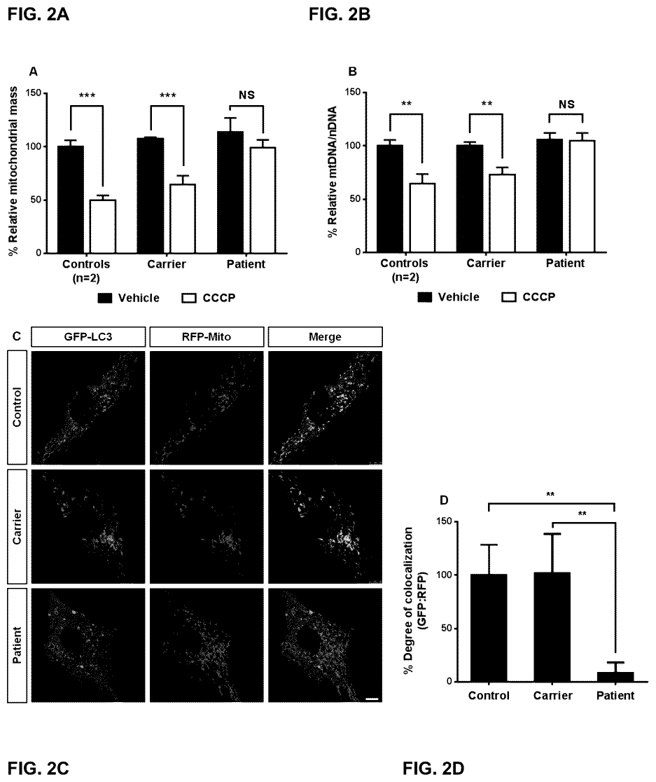

FIG. 2 shows that (A) citrate synthase activity was significantly reduced in the controls and the carrier cells compared to the vehicle-treated counterparts, but not in the patient cells. (B) DNA was isolated from vehicle-treated cells (black bars) or cells treated with 10 μM CCCP (white bars) for 24 hours, followed by mitochondrial DNA quantification using quantitative real-time PCR. The relative amount of mitochondrial DNA (mtDNA) to nuclear DNA (nDNA) was significantly decreased after CCCP treatment in the controls and the carrier cells, but not in the patient cells. N.S; not significant, **; p<0.01, ***; p<0.001 in two-tailed Student's t-test. (C) Fibroblasts expressing GFP-LC3 (an autophagosomal marker, green fluorescence in the left panel) and RFP-Mito (a mitochondrial marker, red fluorescence in the middle panel) were treated with 20 μM CCCP for 4 hours. A high degree of co-localisation between GFP-LC3 and RFP-mito (yellow puncta in the right panel) was observed in the control and the carrier cells, indicating elevated mitophagy, but not in the patient cells. Scale bar: 10 μm (D) Degree of co-localisation was calculated from 50 individual cells. The patient cells displayed a significantly low degree of co-localisation while the carrier cells showed a similar degree, when compared to the control. **; p<0.01 in one-way ANOVA followed by post hoc Tukey's HSD multiple comparison test.

### Example 3. Lack of Compensation for Parkin Function in the Process of Mitophagy and Aberrant Activation of Autophagy in the Carrier Cells

In order to confirm a lack of compensation on the function of Parkin in mitophagy in carrier cells, mitochondrial recruitment of Parkin and ubiquitination of Mitofusin 2 (Mfn2) upon CCCP treatment was assessed.

### Methods

### Mitochondrial Isolation

Isolation of mitochondria from fibroblasts was performed using a protocol employing a standard Dounce homogenizer and Mannitol-Sucrose-EDTA (MSE) buffer (25 mM mannitol, 75 mM sucrose, 100 mM EDTA (Sigma)). Briefly, cells were collected by trypsinisation and resuspended in 3 mL of cold MSE buffer. Cells were lysed using a motorised Dounce homogeniser (Kika Labortechnik, Staufen, Germany). An additional 3 ml of MSE buffer was added to the homogenate, followed by centrifugation at 600×g for 10 minutes at 4° C. The mitochondria containing supernatant was then further centrifuged at 12,000×g for 15 minutes at 4° C. to collect mitochondria. After centrifugation, the supernatant (“cytosolic fraction”) was reserved and concentrated through a centrifugal protein concentrator with 9 kDa molecular weight cut-off (Thermo Scientific), according to the manufacturer's instructions. Meanwhile, the pellet containing mitochondria (“mitochondrial fraction”) was washed twice with 1 ml MSE buffer and finally resuspended in 60 μL lysis buffer (CelLytic M Cell Lysis Reagent supplemented with protease inhibitors cocktail (Sigma)).

### Western Blotting Analysis

Protein expression was determined by Western blotting using the XCell SureLock Mini-Cell Electrophoresis System and XCell II Blot Module (Invitrogen). Briefly, 20 to 30 μg of either total cell lysates or mitochondrial/cytosolic fractions were resolved using NuPAGE Novex 4%-12% Bis-Tris SDS/polyacrylamide gels (Invitrogen) and transferred to a polyvinylidene fluoride (PVDF) membrane (Thermo Scientific). The proteins blotted in the membrane were then probed with a sequential application of protein-specific primary antibodies and horseradish peroxidise-conjugated secondary antibodies. Antibodies used in the assay are detailed in Table 2 below. Chemiluminescence was developed using SuperSignal West Pico or Femto Chemluminescent Substrate (Thermo Scientific) and detected using LAS4000 (Fujifilm, Tokyo, Japan).

TABLE 2
Antibodies used in Western blotting assays

| Antibodies | Suppliers | Dilution | Condition |
|---|---|---|---|
| Parkin | Cell Signalling Technology, Inc., Denver, MA, USA | 1:1000 | 1% skim milk 0.05% TBST, 4° C. 16 hours |
| Mfn2 | Abcam, Cambridge, MA, USA | 1:2000 | 1% skim milk 0.05% TBST, 4° C. 16 hours |
| LC [symbol] | Medical & Biological Laboratories Co., Ltd., Nagoya, Japan | 1:1000 | 1% skim milk 0.05% TBST, 4° C. 16 hours |
| Nix | Abcam, Cambridge, MA, USA | 1:1000 | 5% skim milk 0.05% TBST, 4° C. 16 hours |
| VDAC | Cell Signalling Technology, Inc., Denver, MA, USA | 1:2000 | 5% skim milk 0.05% TBST, RT 1 hour |
| β-actin | Sigma. St. Louis, MO, USA | 1:5000 | 5% skim milk 0.05% TBST, 4° C. 16 hours |
| Anti-mouse IgG | Bio-Rad, Hercules, CA, USA | 1:5000 | 5% skim milk 0.05% TBST, RT 1 hour |
| Anti-rabbit IgG | Sigma, St. Louis, MO, USA | 1:5000 | 5% skim milk 0.05% TBST, RT 1 hour |

TBST, Tris buffered saline with Tween 20.
RT, room temperature [symbol]
indicates data missing or illegible when filed

### RNA Extraction and Quantitative Real Time RT-PCR Analysis

Total RNA from fibroblasts was prepared using the RNeasy Mini Kit (Qiagen) according to the manufacturer's instructions and then reverse-transcribed into cDNA with the SuperScript III First-Strand Synthesis System (Invitrogen) following the manufacturer's instructions. The resulting cDNAs were used to determine gene expression in a quantitative real time RT-PCR (qRT-PCR) using QuantiTect SYBR Green PCR Kit (Qiagen) on the Rotor Gene 6000 (Qiagen) according to the manufacturer's instructions. The primers used in the reaction are listed in Table 3 below.

TABLE 3
Primers used for qRT-PCR analysis

| | | | | Amplicon | |
|---|---|---|---|---|---|
| Gene | RefSeqID | Forward primer | Reverse primer | (bp) | Reference |
| Nix | NM_004331 | TTGGATGCACAACATGAATCAGG | TCTTCTGACTGAGAGCTATGGTC | 140 | 1 |
| GABARAP-L1 | NM_031412 | GACGCCTTATTCTTCTTTGTC | CATGATTGTCCTCATACAGTTG | 79 | 2 |
| GABARAP-L2 | NM_007285 | GTTTGTGGATAAGACAGTCC | GAAGCCAAAAGTGTTCTCTC | 118 | 3 |
| PINK1 | NM_032409.2 | TTCCCCTTGGCCATCAAGA | ACCAGCTCCTGGCTCATTGT | 86 | 4 |
| β-actin | AB004047 | GTCCTCTCCCAAGTCCACAC | GGGAGACCAAAAGCCTTCAT | 188 | |

¹PrimerBank ID# 47078259c2
²KiCqStartprimers, ID# H_GABARAPL 1_3, Sigma, St. Louis, MO, USA
³KiCq Startprimers. ID# H_GABARAPL2_2 Sigma, St. Louis, MO, USA
⁴Seibler, P., et al., 2011. Mitochondrial Parkin recruitmentis impairedin neurons derived from mutant
PINK 1 induced pluripotent stem cells. J. Neurosci. 31, 5970-5978.

### Results

Upon exposure to CCCP, Parkin was highly accumulated in the mitochondrial fraction of the control cells while the protein in the cytosolic fraction was reduced, indicating mitochondrial recruitment of Parkin. In addition, the control cells displayed an increase in the ubiquitinated Mfn2 with reduced amount of the non-ubiquitinated form after CCCP, indicating Parkin-mediated ubiquitination and degradation of Mfn2. However, none of these Parkin-related events was observed in the carrier cells upon CCCP treatment, confirming the lack of Parkin function in the process of mitophagy. In addition, the expression level of PINK1 transcripts was not elevated before and after CCCP treatment in the carrier compared to controls, supporting the lack of compensatory activation in the Parkin/PINK1-mediated mitophagy in the carrier cells. In addition, there is a possibility of hyperactive autophagy mediating non-specific degradation of mitochondria. Therefore, this possibility was also tested by monitoring the conversion of LC3-I to LC3-II upon CCCP treatment as an indicator for autophagic function. Furthermore, CCCP has been demonstrated elsewhere to induce autophagy in HeLa cells, HCT116 cells, and MEF at comparable magnitude of activation to rapamycin. In all cell lines examined, a similar increase of LC3-II/β-actin ratio before and after CCCP treatment was detected, indicating normal function of autophagic machinery under basal and/or mitophagy-inducing conditions. Taken together, these results indicate that CCCP-induced mitophagy observed in the carrier cells is not mediated either by PINK1/Parkin pathway or by an aberrant activation of autophagy, suggesting involvement of a Parkin-independent mitophagic pathway.

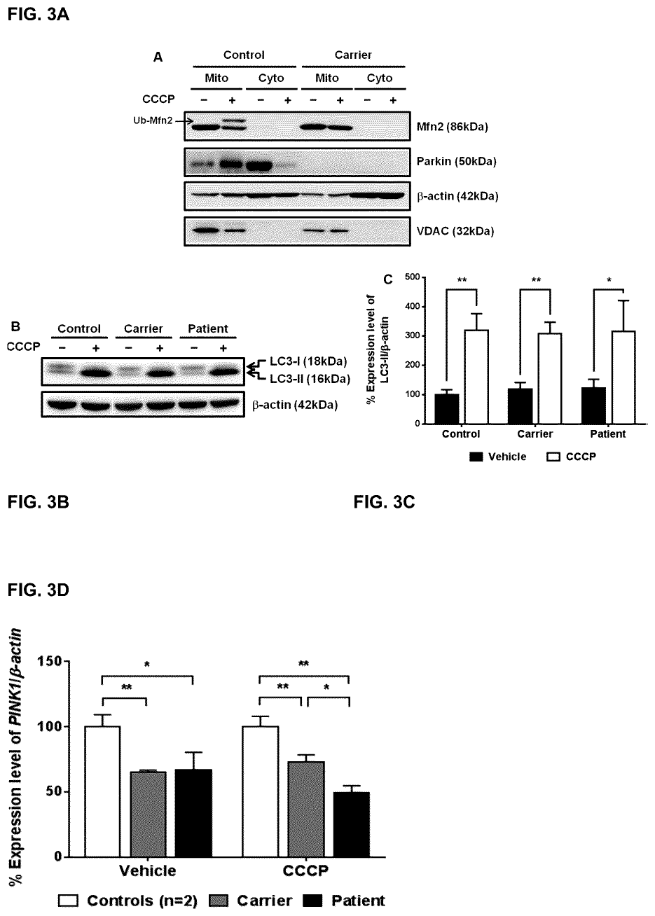

As shown in FIG. 3, (A) Mitochondrial and cytosolic fractions were isolated from fibroblasts treated with either vehicle or 10 μM CCCP for 6 hours and sub-cellular localisation of Parkin and Mfn2 was determined by Western blotting. Quality of the fractionation was confirmed using antibodies against VDAC for mitochondrial faction and β-actin for cytosolic fraction. In the control cells, wild-type 50 kDa Parkin was predominantly found in the cytosolic fraction under basal conditions. Upon exposure to CCCP, the level of Parkin was increased in the mitochondrial fraction and decreased in the cytosolic fraction, indicating translocation of Parkin to mitochondria. The translocation of Parkin to mitochondria was absent in the carrier cells. Reduced expression of the non-ubiquitinated form of Mfn2 and the presence of the ubiquitinated form (Ub-Mfn2) were observed in the control, but not in the carrier cells after exposure to CCCP. Mito: mitochondrial fraction; Cyto: cytosolic fraction; Mfn2: Mitofusin 2; VDAC, Voltage-dependent anion channel. (B-D) Fibroblasts were cultured under basal conditions or treated with 10 μM CCCP for 6 hours and proteins were then collected. (B) Expression of LC3-I and LC3-II in the control, carrier, and patient cells was determined by Western blotting and the bands were quantified using densitometry. β-actin (42 kDa) was used as a loading control. (C) Levels of LC3-II/β-actin ratio were significantly increased upon CCCP treatment compared to untreated groups; however, there is no difference between the cell lines. CCCP induced conversion of LC3-I to LC3-II. *; p<0.05 and **; p<0.01 in two-tailed Student's t-test. (D) Expression of PINK1 was decreased in both carrier and patient cells before and after CCCP treatment, when compared to controls. *; p<0.05 and **; p<0.01 in one-way ANOVA followed by post hoc Tukey's HSD multiple comparison test.

### Example 4. Expression Levels of Nix and GABARAP-L1 are Elevated in the Carrier Cells

In order to assess whether Nix-mediated mitophagy was responsible for the increase in mitophagy induced by CCCP in the carrier cells, expression levels of Nix, GABARAP-L1 and GABARAP-L2 under basal and CCCP-treated conditions were assessed using qRT-PCR.

### Methods

Fibroblasts were cultured under basal conditions or treated with 10 μM CCCP for 6 hour before the extraction of total RNA and cDNA synthesis. Expression of Nix, GABARAP-L1 and GABARAP-L2 was determined by qRT-PCR.

### Results

The expression of Nix was comparable between the controls and the carrier cells under basal conditions, but it was significantly increased in the carrier cells (p<0.01) upon induction of mitophagy by CCCP. The carrier cells also showed an elevated level of GABARAP-L1 but reduced expression of GABARAP-L2 when compared to controls under both conditions. On the other hand, the expression of these genes was found to remain significantly low in the patient cells when compared to controls cells even after CCCP treatment. Taken together, these results indicate the induction of Nix by CCCP treatment and a high expression level of its binding partner GABARAP-L1 in the carrier cells, suggesting their involvement in the alternative mitophagy.

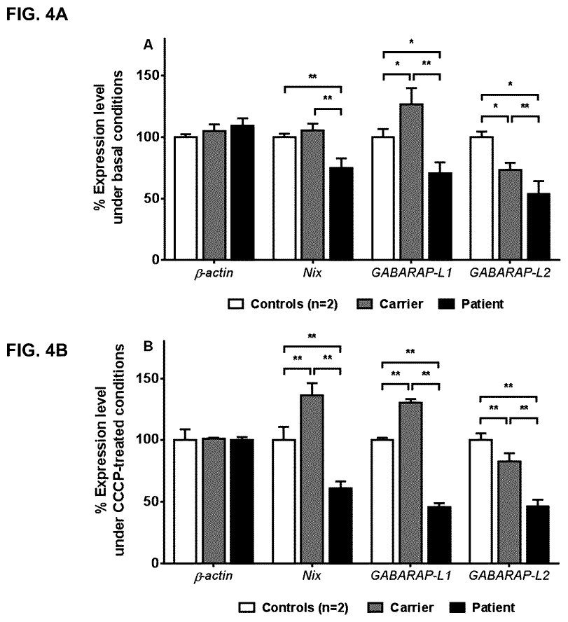

FIG. 4 shows (A) under basal conditions, expression of Nix was similar between the controls and the carrier cells but significantly reduced in the patient cells. Elevated level of GABARAP-L1 was observed in the carrier cells when compared to the control and the patient cells. Expression of GABARAP-L2 was significantly decreased in both carrier and patient cells when compared to the controls. (B) In CCCP-treated conditions, the carrier cells showed a significantly high expression of Nix and GABARAP-L1, but not GABARAP-L2, when compared to controls. Expression of Nix, GABARAP-L1 and GABARAP-L2 remained significantly reduced in the patient cells when compared to controls and carrier cells. *; p<0.05 and **; p<0.01 in one-way ANOVA followed by post hoc Tukey's HSD multiple comparison test.

### Example 5. Knockdown of Nix Impairs CCCP-Induced Mitophagy in the Carrier Cells

In order to confirm its involvement in CCCP-induced mitophagy we silenced Nix using siRNA and assessed change in CCCP-induced mitophagy.

### Methods

### siRNA-Mediated Nix Knockdown

Knockdown of Nix in fibroblasts was achieved using Dharmacon ON-TARGET plus SMART pool-Human BNIP3L (refer to as Nix siRNA; Thermo Scientific, #L-011815-00-0005) and DharmaFECT1 siRNA Transfection Reagent (Thermo Scientific, #T-2001-01) following the manufacturer's instructions. ON-TARGET plus Non-Targeting siRNA #1 (refer to as scramble siRNA; Thermo Scientific, #D-001810-01-05) was used as a negative control.

Gene knockdown was confirmed at the mRNA and protein levels using qRT-PCR and Western blotting respectively, 48 hours post transfection. Greater than 95% reduction in the target mRNA level was regarded as successful knockdown.

### Results

The expression level of Nix at 48 hours post transfection of siRNA was dramatically reduced at the mRNA level (>95%) and at the protein level, indicating a successful knockdown. Following exposure to CCCP, the cells transfected with scramble siRNA displayed a significant reduction of mitochondrial mass measured by citrate synthase activity (63.0% reduction in the control cells, p<0.001 and 30.1% in the carrier cells, p<0.05 in comparison to the respective vehicle controls), indicating normal mitophagy. However, transfection of Nix siRNA abrogated the reduction of mitochondrial mass in the carrier, but not in the control cells (47.6% reduction in the control cells, p<0.01 and 8.5% in the carrier cells, p=0.15). A similar result was obtained from the assessment of mtDNA content in the cells transfected with scramble siRNA (20.7% reduction in the control cells, p<0.001 and 33.0% in the carrier, p<0.05) and Nix siRNA (36.0% reduction in the control cells, p<0.01 and 8.1% in the carrier cells, p=0.39). In addition, the carrier cells transfected with Nix siRNA showed a marked reduction in co-localisation of GFP-LC3 and RFP-mito compared to the cells transfected with scramble siRNA upon induction of mitophagy by CCCP, while a similar low degree of co-localisation between the carrier cells transfected with scramble and Nix siRNA was observed under basal conditions (data not shown). Quantification of co-localisation revealed a significant reduction in the Nix siRNA-transfected carrier cells (63.0% reduction, p<0.001) when compared to the respective scramble siRNA cells, demonstrating impairment of CCCP-induced mitophagy. Taken together, these results indicate that Nix facilitates CCCP-induced mitophagy in the carrier cells with Parkin loss-of-function.

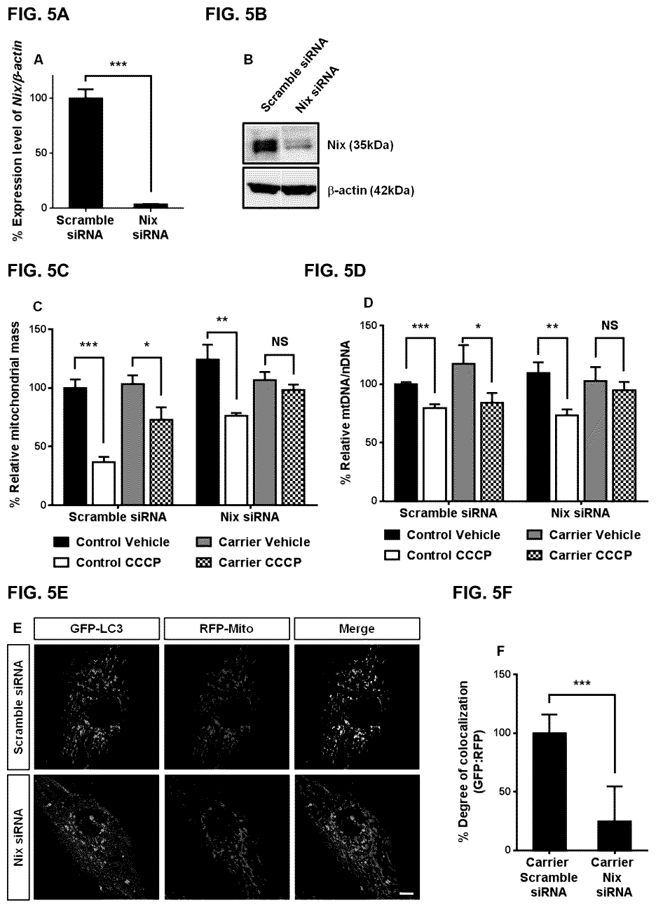

FIG. 5 shows successful knockdown of Nix was confirmed at mRNA level (A) and at protein level (B). (C and D) Cell lysates and DNA were prepared from vehicle-treated cells or cells treated with 10 μM CCCP for 24 hour after Nix knockdown. (C) Mitochondrial mass was measured using citrate synthase assay. Upon CCCP treatment, citrate synthase activity was significantly reduced in the cells treated with scramble siRNA and in the Nix siRNA-treated control cells, but not in the carrier cells treated with Nix siRNA. (D) Mitochondrial DNA quantification showed that the relative amount of mtDNA to nDNA was significantly decreased after CCCP treatment in the scramble siRNA-treated cells and in the Nix siRNA-treated control cells, but not in the carrier cells treated with Nix siRNA. NS; Not significant, *; p<0.05, **; p<0.01, ***; p<0.001 in two-tailed Student's t-test. (E) Fibroblasts expressing GFP-LC3 (an autophagosomal marker, green fluorescence in the left panel) and RFP-Mito (a mitochondrial marker, red fluorescence in the middle panel) were treated with 25 nM of either scramble or Nix siRNA. At 72 hours post-siRNA transfection, cells were incubated with 20 μM CCCP for 4 hours to induce mitophagy. Under the CCCP treatment, a high degree of co-localisation between GFP-LC3 and RFP-Mito (yellow puncta in the right panel) was observed in the carrier cells treated with scramble siRNA, indicating elevated mitophagy while Nix siRNA impaired mitophagy in the carrier cells. Scale bar: 10 μm. (F) Degree of co-localisation were calculated from 50 individual cell images. Under CCCP treatment, the Nix siRNA-treated carrier cells depicted a significantly low degree of co-localisation when compared to the scramble siRNA-treated counterpart. ***; p<0.001 in two-tailed Student's t-test.

### Example 6. Specific Induction of Nix Expression in Patient Cells Restores Mitophagy

In order to confirm the relationship between of Nix expression and CCCP-induced mitophagy we increased Nix expression in cells having deficient Nix expression with respect to controls and the carrier cells and assessed change in CCCP-induced mitophagy.

### Methods

### Increased Expression of Nix

In order to assess the effects of phorbol myristate acetate (PMA) on Nix expression, control cells and patient cells (“proband”) were exposed to PMA (10 nM or 20 nM) for 24 hours. Cells were harvested after 24 hours and expression of Nix and GABARAP-L1 protein was determined by Western blotting as outlined above.

The functional effects of the induction of Nix expression on mitophagy were assessed using methods outlined above. Patient cells were co-treated with CCCP and PMA for 24 hours and mitophagy was examined via measurement of the mitochondrial mass by citrate synthase activity and the mtDNA content by quantitative real-time PCR. Cells used in this assay include “patient” cells as hereinbefore described and cells isolated from an individual with PD identified with homozygous PINK1 mutations at c.1309T>G (p.W437G) “PINK1mut”.

### Results

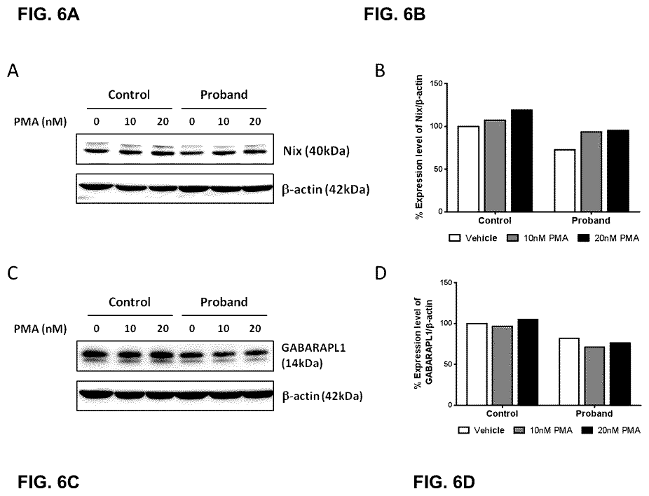

FIG. 6 shows expression of Nix (A-B) and GABARAP-L1 (C-D) in the control and patient cells was determined by Western blotting and the bands were quantified using densitometry. β-Actin (42 kDa) was used as a loading control. Levels of Nix/β-actin ratio were increased upon PMA treatment compared to the vehicle control (A-B). There was no increase in GABARAP-L1 expression upon exposure to PMA(C-D).

In accordance with the specific induction of Nix expression in patient cells by exposure to PMA, cells that were administered 10 nM PMA in the presence of CCCP demonstrated a significant reduction in mitochondrial mass and mitochondrial DNA. Assessment of mitochondrial mass via the citrate synthase assay, demonstrated that administration of PMA to control cells did not impact CCCP-induced mitophagy. However, in cells lacking functional Parkin and having impaired mitophagy in response to CCCP treatment, PMA significantly reduced mitochondrial mass (79.82%±4% vs 103.7%±2% for CCCP+PMA vs CCCP, vehicle control as 100%, p<0.01). Similarly, mitochondrial DNA was significantly reduced when cells were administered PMA (77.06±4% vs 93.97±5% for CCCP+PMA vs CCCP, vehicle control as 100%, p<0.05) similar to the levels observed in control cells (72.79±6%).

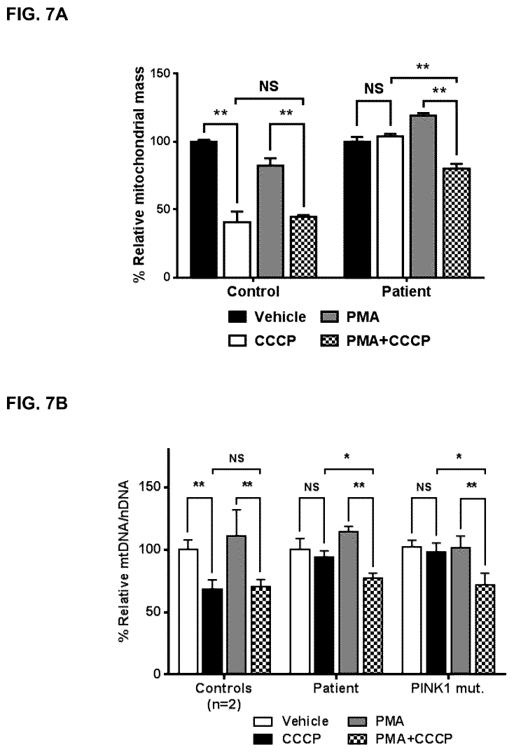

FIG. 7 shows induction of Nix by PMA restores mitophagy in patient cells. (A) Cell lysates were prepared from vehicle-treated cells (black bars), CCCP-treated cells (white bars), PMA-treated cells (grey) and cells treated with 10 nM PMA and 10 μM CCCP (checker) for 24 hours, followed by measurement of citrate synthase activity. Co-treatment of PMA and CCCP significantly reduced the citrate synthase activity in the patient cells and “PINK1mut” that was not otherwise observed upon CCCP treatment alone. (B) DNA was isolated from vehicle-treated cells (black bars), CCCP-treated cells (white bars), PMA-treated cells (Grey) and cells treated with 10 nM PMA and 10 μM CCCP (Checker) for 24 hours, followed by mitochondrial DNA quantification using quantitative real-time PCR. The relative amount of mitochondrial DNA (mtDNA) to nuclear DNA (nDNA) was significantly decreased after PMA and CCCP co-treatment in the patient and PINK1 mut cells. NS; not significant *; p<0.05 and **; p<0.01 in one-way ANOVA followed by post hoc Tukey's HSD multiple comparison test.

These results indicate that administration of an agent that increases expression of Nix is able to rescue impaired mitophagy associated with Parkin loss-of-function.

### Example 7. Knockdown of Nix in Patient Cells and Cells Carrying a Mutation in PINK1 Abrogates Restoration of CCCP-Induced Mitophagy Achieved by Specific Induction of Nix

In order to assess the specificity of the observed restoration of mitophagy in patient cells treated with an agent which induces expression of Nix, mitophagy was assessed in cells isolated from an individual carrying compound heterozygous mutations in parkin and cells isolated from an individual carrying a homozygous mutation in PINK1.

### Methods

### siRNA-Mediated Knockdown of Nix

In order to assess the specificity of phorbol myristate acetate (PMA)-induced restoration of mitophagy, control cells, cells isolated from an individual carrying compound heterozygous mutations in parkin (“patient”) and cells isolated from an individual carrying a homozygous mutation in PINK1 (“PINK1”) were subjected to siRNA-mediated knockdown of Nix as outlined in Example 5 above. Briefly, cells were exposed either to non-targeting siRNA (Scramble siRNA) or siRNA targeting Nix (Nix siRNA) followed by co-treatment with CCCP and PMA for 24 hours.

Cells were harvested after 24 hours and the effect of knock-down of Nix on mitophagy was assessed via measurement of the mtDNA content by quantitative real-time PCR as outlined above.

### Results

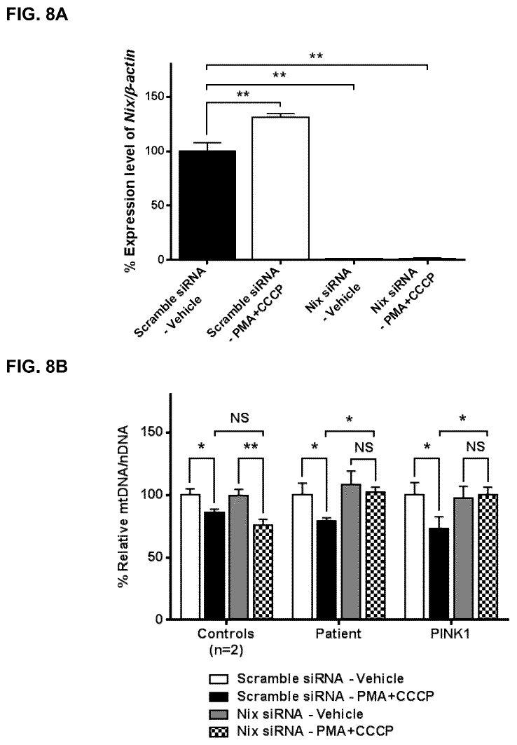

FIG. 8(A) shows expression of Nix following treatment of cells with Scramble siRNA or siRNA targeting Nix. Successful knockdown of Nix was achieved. FIG. 8(B) also shows Patient and PINK1 cells treated with Nix siRNA showed no significant decrease in mtDNA after PMA and CCCP co-treatment when compared to the respective Nix siRNA-vehicle-treated cells. NS; not significant and *; p<0.05 in one-way ANOVA followed by post hoc Tukey's HSD multiple comparison test.

The absence of a decrease in the amount of mtDNA relative to nDNA in Nix-silenced cells by a sequential treatment of PMA and CCCP demonstrates that the PMA-associated restoration of mitophagy in cells lacking functional parkin or PINK1 is Nix-specific.

These results confirm that restoration of mitophagy by PMA in cells lacking the PINK1/Parkin mitophagic pathway is indeed mediated by Nix.

### Example 8. Over-Expression of Nix Restores CCCP-Induced Mitophagy in Patient Cells

In order to confirm the relationship between of Nix expression and CCCP-induced mitophagy we overexpressed Nix in patient cells lacking functional parkin (and having deficient Nix expression with respect to controls and the “carrier” cells) and assessed change in CCCP-induced mitophagy.

### Methods

### Over-Expression of Nix

Wild-type Nix cDNA (NM_004331) in pCMV6-Nix (Origene; #RC203315) was subcloned into a pER4 lentiviral vector containing FLAG tag. Lentivirus for the expression of Nix-FLAG was produced using the Lenti-X HTX Lentiviral Packaging system (Clontech, Mountain View, CA, USA) and Lipofectamine 2000 (Invitrogen, Carlsbad, CA, USA) according to the manufacturer's instruction. The media containing lentivirus was collected at 48 and 72 hrs post-transfection followed by concentration step using the Lenti-X concentrator (Clontech) before measurement of viral titre. Fibroblasts were transduced with either an empty lentiviral vector (pEmpty) or a lentiviral Nix-FLAG vector (pNix-FLAG) with a ratio of 10 infectious units of lentivirus per cell in the presence of 4 μg/mL polybrene for 24 hrs and used for subsequent experiments.

The functional effects of Nix over-expression on mitophagy were assessed using methods outlined above. Briefly, the cells transduced with lentivirus were treated with CCCP or vehicle for 24 hours and mitophagy was examined via measurement of mtDNA content by quantitative real-time PCR, and degree of co-localisation of autophagosomes and mitochondria as outlined in Example 2.

### Results

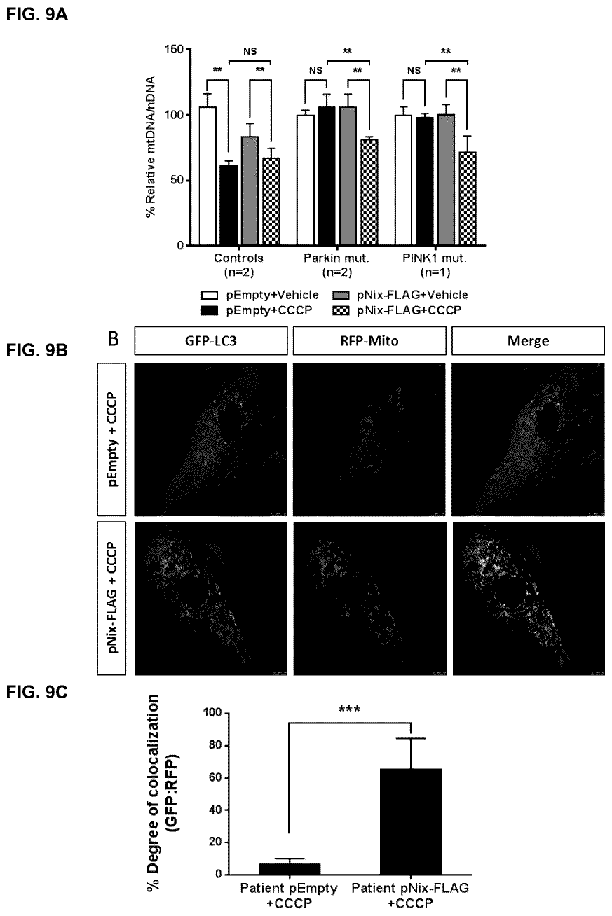

FIG. 9 shows over-expression of Nix restores CCCP-induced mitophagy in cells lacking functional parkin (including the patient cells; “Parkin mut.”) and in cells isolated from an individual carrying a homozygous mutation in PINK1 (“PINK1 mut.”). Fibroblasts were transduced with either lentivirus containing an empty vector (pEmpty) or Nix-FLAG vector (pNix-FLAG). DNA was isolated from vehicle-treated cells and CCCP-treated cells, followed by mitochondrial DNA quantification using quantitative real-time PCR. (A) In Parkin and PINK1 mutants expressing Nix-FLAG, the relative amount of mitochondrial DNA (mtDNA) to nuclear DNA (nDNA) was significantly decreased after CCCP treatment when compared to the vehicle-treated cells. NS; not significant **; p<0.01 in one-way ANOVA followed by post hoc Tukey's HSD multiple comparison test. (B) Patient cells expressing GFP-LC3 (Green) and RFP-Mito (Red) that were transduced with lentivirus were treated with 20 μM CCCP for 4 hr. Co-localisation of autophagosomes and mitochondria (yellow puncta in the right panel) was observed in the patient cells expressing Nix-FLAG, indicating activation of mitophagy, but not in the patient cells expressing the empty vector. Scale bar: 10 μm. Co-localisation rates were calculated from 50 individual cell images using Leica Application Suite Advance Fluorescence (LAS AF) software (C). Following CCCP treatment, patient cells expressing Nix-FLAG displayed a significantly high co-localisation rate compared to the empty vector-transduced cells. ***p<0.001 in two-tailed Student's t-test.

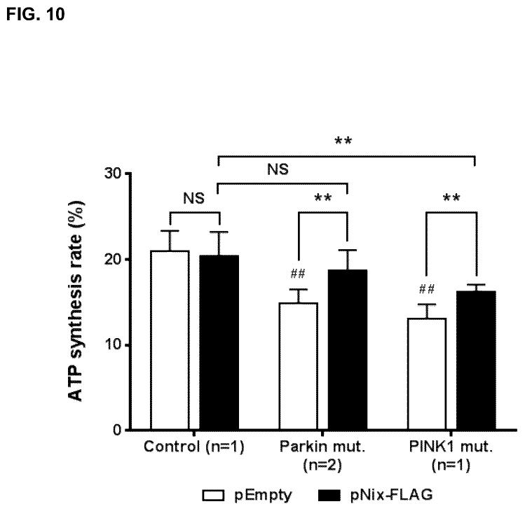

FIG. 10 shows over-expression of Nix improves mitochondrial function in Parkin and PINK1 mutant fibroblasts. Parkin and PINK1 mutant cells were transduced with either empty lentiviral vector (pEmpty) or Nix-FLAG vector (pNix-FLAG) and cultured for 72 hr. Mitochondrial ATP synthesis rate was measured spectrophotometrically in the presence of malate and pyruvate in digitonin-permeabilised cells. Cells over-expressing Nix showed a significant increase in ATP synthesis rate when compared to the empty vector-transduced cells. NS; not significant, **; p<0.01 as indicated in the graph and ##; p<0.01 in mutant cells expressing pEmpty vs control cells expressing pEmpty cells in one-way ANOVA followed by post hoc Tukey's HSD multiple comparison test.

These results demonstrate that administration of an agent which augments expression of Nix to cells which lack functional parkin or PINK1, and which display impaired mitophagy and mitochondrial function, restores mitophagy and mitochondrial function.

1. A method for the treating or reducing the probability of developing a neurodegenerative disorder in a subject, comprising the steps of:
a. identifying a subject as having impaired parkin-mediated mitophagy, and
b. administering to the subject a therapeutically effective amount of an agent that increases Nix-mediated mitophagy in a cell, wherein the agent increases the biological activity or expression of a Nix polypeptide and/or a GABARAP-L1 polypeptide in a cell, and wherein the agent comprises a Nix polypeptide and/or a GABARAP-L1 polypeptide; an expression vector encoding a Nix polypeptide and/or a GABARAP-L1 polypeptide; or phorbol myristate acetate (PMA).

2. The method according to claim 1, wherein the agent comprises an expression vector encoding a Nix polypeptide thereof.

3. The method according to claim 1, wherein the agent comprises a Nix polypeptide.

4. The method according to claim 1, wherein the agent comprises phorbol myristate acetate (PMA).

5. The method according to according claim 1, wherein the cell is a neuron or a neuronal precursor.

6. The method according to claim 1, wherein the neurodegenerative disorder is associated with mitochondrial dysfunction.

7. The method according to claim 1, wherein the neurodegenerative disorder is selected from the group comprising Parkinson's disease, Alzheimer's disease, Lewy body dementia, Creutzfeldt-Jakob disease, Huntington's disease, multiple sclerosis or amyotrophic lateral sclerosis.

8. The method according to claim 6, wherein the neurodegenerative disorder is Parkinson's disease.

9. The method according to claim 1, wherein said subject possesses a mutation in parkin and/or PINK1.

10. The method according to claim 1, wherein said subject has a reduced expression of parkin and/or PINK1.
